(19) United States
(12) Patent Application Publication
(10) Pub. No.: US 2019/0338272 A1
(43) Pub. Date: Nov. 7, 2019

(54) MASSIVELY MULTIPLEXED HOMOLOGOUS TEMPLATE REPAIR FOR WHOLE-GENOME REPLACEMENT

(71) Applicant: CHRISTOPHER BRADLEY, BROOKLYN, NY (US)

(72) Inventor: CHRISTOPHER BRADLEY, BROOKLYN, NY (US)

(21) Appl. No.: 16/092,761

(22) PCT Filed: Nov. 17, 2017
(86) PCT No.: PCT/US17/62430

§ 371 Date: Oct. 11, 2018

Related U.S. Application Data
(60) Provisional application No. 62/424,195, filed on Nov. 18, 2016.
(60) Provisional application No. 62/567,744, filed on Oct. 3, 2017.
(60) Provisional application No. 62/567,259, filed on Oct. 3, 2017.
(60) Provisional application No. 62/554,738, filed on Sep. 6, 2017.

(51) Int. Cl. C12N 15/10; A61K 38/46; C12N 15/82; A61K 31/7105

(52) U.S. Cl. CPC C12N 15/102; A61K 38/465; C12N 2800/80; A61K 31/7105; C12N 2310/20; C12N 15/8213

(57) ABSTRACT

Disclosed are systems and methods for whole-genome replacement through a massively multiplexed homologous template repair process. Disclosed aspects include a method of substantially changing a DNA sequence of an organism, the method including one or more of the following steps: determining a desired DNA sequence, the desired DNA sequence being a DNA sequence to which it is desired that the DNA sequence of the organism be substantially changed; preparing a treatment configured to cause the organism DNA sequence to be substantially changed to the desired DNA sequence; applying the treatment to the organism; wherein the treatment is configured to cause the organism DNA sequence to be substantially changed to the desired DNA sequence by causing, at each of multiple sites in the organism DNA sequence, genetic code at the site to be substantially changed to genetic code at a responding site in the desired DNA sequence; each of the multiple sites in the organism DNA sequence is a respective sub-sequence of the organism DNA sequence; applying the treatment includes delivering to the organism at least one dose; and each dose includes respective change agent material that causes the changing of the genetic code at a respective plurality of the multiple sites. In certain embodiments, the DNA sequence of the organism is a whole-genome DNA sequence of the organism, and the desired DNA sequence is substantially the organism's germline whole-genome DNA sequence, an intentionally modified version of the organism's germline whole-genome DNA sequence, or a whole-genome DNA sequence of another organism.

### CROSS REFERENCE TO RELATED APPLICATIONS

The present application claims priority from the following U.S. Provisional Applications, the entire disclosures of which, including but not limited to any and all cited references, are incorporated herein by reference: U.S. Provisional Application Nos. 62/424,195 (filed Nov. 18, 2016), 62/508,259 (filed May 18, 2017), 62/554,738 (filed Sep. 6, 2017), and 62/567,744 (filed Oct. 3, 2017).

### FIELD OF THE INVENTION

The present invention relates generally to systems and methods for genetic modification, and more particularly to systems and methods for whole-genome replacement.

### BACKGROUND OF THE INVENTION

Ever since discovering the structure and function of DNA and successfully sequencing the genomes of humans and other animals, scientists have been able to slowly tease apart the extremely complex cause and effect relationships at the heart of life itself. The deeper one looks, however, the deeper the complexities and interconnections seems to become.

Diseases that epitomized the importance of genomics, such as cancer, have revealed themselves to be caused by much more than a simple on/off switch, or single base pair mutation. Instead, we are discovering that cancer is caused by a wide spectrum of different cascading mutations and ultimately different and constantly evolving diseases. So far, the same has been shown to be the case with aging. While no one has discovered a definitive cause of aging in humans or other animals, the current evidence points to aging being an emergent phenomenon resulting from failures of a large number of different pathways and processes.

One thing, however, is clear: increases in the amount and rate of mutagenesis, whether by external or internal factors, contribute greatly to a large number of genetic pathologies. This is thought to encompass everything from cancer and degenerative disease, to aging itself.

Accordingly, there is a need to reverse the damage and mutagenesis present in a cell's genome, which may help cure and prevent diseases that emerge from their accumulation.

### SUMMARY OF THE INVENTION

Accordingly, one or more aspects of the present invention include, but are not limited to, providing systems and methods for whole-genome replacement.

One or more aspects of the systems and methods for whole-genome replacement described herein are sometimes referred to herein, not by way of limitation but rather for purposes of convenience, as massively multiplexed homologous template repair (“MMHTR”).

It is contemplated that the systems and methods of the present invention can be used to replace, in every cell in an organism's body, the current, mutagenic, whole-genome DNA sequence of the cell with a pre-mutagenic copy of the organism's whole-genome DNA sequence. It is contemplated that such a complete replacement will “turn back the clock” on the organism's genome, removing any accumulated damage, thus returning each cell, and therefore the entire organism, to a germline state. Preferably, only the nuclear DNA sequence would be replaced or “refreshed”, and the epigenetic properties of the genome, including any gene expression patterns, would not be disrupted or altered.

It is anticipated that by removing accumulated mutagenesis, the cell will cease to display any of the phenotypic traits associated with the mutagenesis, such as, for example, cancer, neurodevelopmental disorders, development disorders, and other disorders. Additionally, to the extent that accumulated mutagenesis contributes to cellular aging, its removal is anticipated to have a substantial phenotypic effect on age-related properties of the cells of the organism and the organs and systems to which the cells belongs.

In a preferred embodiment, a method of the present invention includes a method of substantially changing a DNA sequence of an organism, the method including but not limited to one or more of the following steps: determining a desired DNA sequence, the desired DNA sequence being a DNA sequence to which it is desired that the DNA sequence of the organism be substantially changed; preparing a treatment configured to cause the organism DNA sequence to be substantially changed to the desired DNA sequence; applying the treatment to the organism; wherein the treatment is configured to cause the organism DNA sequence to be substantially changed to the desired DNA sequence by causing, at each of multiple sites in the organism DNA sequence, genetic code at the site to be substantially changed to genetic code at a corresponding site in the desired DNA sequence; each of the multiple sites in the organism DNA sequence is a respective sub-sequence of the organism DNA sequence; applying the treatment includes delivering to the organism at least one dose; and each dose includes respective change agent material that causes the changing of the genetic code at a respective plurality of the multiple sites.

Preferably, the DNA sequence of the organism is a whole-genome DNA sequence of the organism.

Preferably, the desired DNA sequence is substantially at least one of a germline whole-genome DNA sequence of the organism, a pre-mutagenic whole-genome DNA sequence of the organism, a global average whole-genome DNA sequence of the organism, a whole-genome DNA sequence of another organism, and a whole-genome DNA sequence that is an intentionally modified version of a germline whole-genome DNA sequence of the organism.

In certain embodiments of the present invention, the desired DNA sequence is a desired whole-genome DNA sequence, the desired sequence having a plurality of nucleotides each at a respective nucleotide position in the desired sequence, the desired sequence being determined by a method including but not limited to one or more of the following steps: determining a necessary sample number, the necessary sample number being equal to a number of whole-genome DNA sequence samples, each having a plurality of nucleotides each at a respective nucleotide position in the sample, each sample being from a different cell of the organism, that must be sequenced to establish that, for each of the nucleotide positions in the desired sequence, the nucleotide at the corresponding nucleotide position in a majority of the samples is the nucleotide desireable for the nucleotide position in the desired sequence; sequencing the necessary sample number of whole-genome DNA sequence samples to determine, for each of the samples, a respective sample sequence; and establishing that, for each nucleotide position in the desired sequence, the nucleotide at the nucleotide position in the desired sequence is the nucleotide at the corresponding nucleotide position in a majority of the samples.

Preferably as to this method of determining a desired whole-genome DNA sequence, in certain embodiments of the present invention, a germline whole-genome DNA sequence of the organism has a plurality of nucleotides each at a respective nucleotide position in the germline sequence, and the nucleotide desireable for the nucleotide position in the desired sequence is the nucleotide in the corresponding nucleotide position in the germline sequence.

Further preferably as to this method of determining a desired whole-genome DNA sequence, in certain embodiments of the present invention, the necessary sample number is equal to a number of the whole-genome DNA sequence samples that must be sequenced to establish that a probability, that for each sample, each nucleotide in its respective nucleotide position in the sample is not the nucleotide at the corresponding nucleotide position in the germline sequence, is less than a desired probability, the desired probability being no greater than one divided by the total number of nucleotides in the germline sequence.

Further preferably as to this method of determining a desired whole-genome DNA sequence, in certain embodiments of the present invention, the desired probability is a product of a first factor and a second factor, the first factor being an error rate of a sequencing method used to sequence at least one of the samples, the second factor being a number no greater than one divided by the total number of nucleotides in the germline sequence.

Preferably as to this method of determining a desired whole-genome DNA sequence, in certain embodiments of the present invention, determining the necessary sample number includes accounting for an error rate of a sequencing method used to sequence at least one of the samples.

Preferably, as to the above described method of substantially changing a DNA sequence of an organism, applying the treatment preferably includes administering an aqueous solution to one or more cells of the organism.

Preferably, administering the solution includes at least one of injecting the solution into the organism adjacent to the one or more cells and intravenously introducing the solution into a bloodstream of the organism.

Preferably, as to the above described method of substantially changing a DNA sequence of an organism, the method further includes but is not limited to one or more of the following steps: obtaining, from each of a plurality of cells of the organism, the cells being randomly chosen from among cells of interest, a DNA sequence from the cell; comparing each of the obtained DNA sequences to the desired DNA sequence; and based on results of the comparison, taking one or more of the following steps: applying the treatment to the organism again, applying to the organism a treatment modified based on results of the comparison, and determining that no additional treatment applications are necessary.

Preferably, as to the above described method of substantially changing a DNA sequence of an organism, the change agent material is useful to treat the organism but not useful to treat any other organism.

Preferably, as to the above described method of substantially changing a DNA sequence of an organism, preparing the treatment preferably includes but is not limited to one or more of the following steps: determining, for each of the multiple sites, at least one targeting sequence; determining, for each of the multiple sites, at least one homology repair template based on at least one characteristic of the at least one targeting sequence for the site; synthesizing the targeting sequences and the homology repair templates; amplifying the targeting sequences and the homology repair templates; post-processing the homology repair templates; packaging the targeting sequences and the homology repair templates into at least one delivery vehicle; post-processing the delivery vehicle, wherein post-processing the delivery vehicle includes at least one of increasing delivery success and decreasing side effects; preparing a solution that includes the change agent material, the change agent material including the post-processed delivery vehicle including the targeting sequences, the post-processed homology repair templates, and at least one of at least one targeted nuclease protein and at least one nucleic acid sequence that expresses at least one targeted nuclease protein; and formulating the dose, the dose including the solution; wherein formulating the dose includes modifying, for treating the organism, the solution as to one or more of the following aspects: concentration, molarity, dosage and content.

Preferably, the change agent material includes a plurality of targeting sequences, a plurality of homologous repair templates based on at least one of the plurality of targeting sequences, and at least one of at least one targeted nuclease protein and at least one nucleic acid sequence that expresses at least one targeted nuclease protein.

Further preferably, as to the above described method of preparing the treatment, preferably the targeting sequences and homology repair templates are determined by, the delivery vehicle is chosen by, and at least one of the at least one targeted nuclease protein and the at least one nucleic acid sequence are chosen by, repeating a process until all desired changes to the organism DNA sequence are expected to result from use of the change agent material in the dose, the process including but not limited to one or more of the following steps: for the dose, establish at least one dose requirement, the at least one dose requirement being based on at least one dose criterion, the at least one dose criterion being one or more of at least one delivery vehicle characteristic, at least one targeted nuclease characteristic, at least one off-target effect characteristic, at least one cutting mechanism characteristic, at least one organism characteristic, and at least one targeting sequence characteristic; select a delivery vehicle and a targeted nuclease that meet the at least one dose requirement to a desired degree; for each of a desired number of base pair sequences of the organism DNA sequence, when the targeted nuclease requires at least one binding site sequence specific to the targeted nuclease, compare the base pair sequence to the at least one binding site sequence; for each of the compared base pair sequences, when the base pair sequence is compatible with the specific at least one binding site sequence, establish as a respective candidate binding site a binding site defined by binding site location information of the base pair sequence, the binding site location information including a start location, an end location, and a base pair letter sequence; for each candidate binding site, using target site selection rules specific to the targeted nuclease, establish as a respective candidate target site a target site defined by target site location information associated with the candidate binding site, the target site location information including a start location and an end location, the rules including one or more of a distance from the candidate binding site and a base pair length; for each candidate target site, using a cutting profile of the targeted nuclease, establish as a respective candidate cut location a location associated with the candidate target site and at which a cut is most likely to occur; for each candidate cut location, establish the candidate cut location as a respective appropriate cut location based on at least one cut location suitability factor, the at least one cut location suitability factor being one or more of a homology repair template maximum size, a homology repair template homology arm size, and a donor sequence maximum size; establish a plurality of homology repair templates by, for each homology repair template, determining a homology repair template target group, the group including at least two cut locations of the appropriate cut locations, the group meeting at least one compatibility requirement with respect to at least one other homology repair template target group; for each appropriate cut location, establish as a respective dose target site the candidate target site associated with the appropriate cut location; and translate each dose target site into a respective targeted nuclease targeting sequence.

Preferably, the process further includes but is not limited to, for at least one homology repair template, mutating or removing any targeted nuclease binding site sequences or donor sequences in the homology repair template.

Preferably, the process further includes but is not limited to, for at least one homology repair template, modifying or replacing a donor sequence to include the desired DNA sequence.

Preferably, the at least one delivery vehicle characteristic is selected from the group consisting of a delivery vehicle size, a delivery vehicle cost, a delivery vehicle ease of ingestion, a delivery vehicle successful payload delivery likelihood, a delivery vehicle delivery route, and a delivery vehicle immunogenicity; the at least one targeted nuclease characteristic is selected from the group consisting of a targeted nuclease targeting specificity, a targeted nuclease targeting consistency, a targeted nuclease binding specificity, a targeted nuclease binding consistency, a targeted nuclease cutting specificity, a targeted nuclease cutting consistency, targeted nuclease cost, a targeted nuclease required binding sequence distribution, a targeted nuclease required binding sequence probability, a targeted nuclease required binding sequence quantity; the at least one off-target effect characteristic is selected from the group consisting of a likelihood of off-target effect occurrence, a likelihood of off-target effect amounts, and a likelihood of off-target cuts; the at least one cutting mechanism characteristic is selected from the group consisting of a cutting mechanism type and a cutting mechanism action; the at least one organism characteristic is selected from the group consisting of an organism nuclease activity amount and an organism nuclease degradation rate; and the at least one targeting sequence characteristic is a targeting sequence length.

Preferably, the at least one compatibility requirement is selected from the group consisting of a homology repair template maximum size, a donor sequence overlap limit, a homology repair template overlap limit, a number of cuts to be made in the organism DNA sequence; a presence of a targeted nuclease binding site within a homology repair template; and a presence of a donor sequence associated with a targeted nuclease binding site within a homology repair template.

Aspects of the present invention are described herein and in the accompanying figures; however, it should be understood that the specific elements and methods in the descriptions are merely examples of elements and methods that can be used to embody and/or implement the described features and processes, and that such broader range of elements and methods are part of the present invention. Therefore, the descriptions herein and in the accompanying figures should be read broadly and understood broadly with respect to such specific elements and methods so as to encompass other elements and methods of such and/or similar and/or related types and/or functions, now known or hereinafter developed, to gain a proper understanding of the broad scope of the present invention.

### BRIEF DESCRIPTION OF THE DRAWINGS

FIG. 1 illustrates a natural accumulation of genetic mutations in the nuclear DNA of a cell over time.

FIG. 2 illustrates a relationship between the number of mutations in a cell and the passage of time.

FIG. 3 illustrates a relationship between the number of mutations in a cell and the passage of time, with the periodic application of change agent material of an embodiment of the present invention, in accordance with an embodiment of the present invention.

FIG. 4A illustrates a preferred method of an embodiment of the present invention, for changing an organism's DNA sequence.

FIG. 4B illustrates a preferred method of an embodiment of the present invention, for determining a desired DNA sequence of an embodiment of the present invention.

FIG. 4C illustrates a preferred method of an embodiment of the present invention, for preparing a treatment of an embodiment of the present invention.

FIG. 4D illustrates a preferred method of an embodiment of the present invention, for determining change agent material of an embodiment of the present invention.

FIG. 5 illustrates an effect of applying, to a mutagenic DNA site, change agent material of an embodiment of the present invention, in accordance with an embodiment of the present invention.

FIG. 6 illustrates a changing state of mutagenesis in DNA of a cell over time and treated with single and then multiple applications of change agent material of an embodiment of the present invention in accordance with an embodiment of the present invention.

FIG. 7 illustrates a changing state of mutagenesis in DNA of a cell prior to being, and thereafter when, treated with multiple doses, including change agent material of the present invention, configured to, in combination with one another, cover the entirety of the organism DNA sequence being treated.

FIG. 8 illustrates an example of an organism DNA sequence to which changes are to be applied in accordance with an embodiment of the present invention.

FIG. 9 illustrates the organism DNA sequence of FIG. 8, with binding sites, of an embodiment of the present invention, being identified.

FIG. 10 illustrates the organism DNA sequence of FIG. 8, with candidate target sites, of an embodiment of the present invention, being identified.

FIG. 11 illustrates the organism DNA sequence of FIG. 8, with candidate cut locations, of an embodiment of the present invention, being identified.

FIG. 12A illustrates the organism DNA sequence of FIG. 8, with appropriate cut locations, of an embodiment of the present invention, and four target selection groups, of an embodiment of the present invention, being identified.

FIG. 12B illustrates the organism DNA sequence of FIG. 8, with alternate target selection groups, of an embodiment of the present invention, being identified.

FIG. 13 illustrates a first homology repair template, of an embodiment of the present invention, created by a first target selection group of an embodiment of the present invention.

FIG. 14 illustrates a second homology repair template, of an embodiment of the present invention, created by a second target selection group of an embodiment of the invention.

FIG. 15 illustrates a third homology repair template, of an embodiment of the present invention, created by the third target selection group of FIG. 14.

FIG. 16 illustrates a fourth homology repair template, of an embodiment of the present invention, created by the fourth target selection group of FIG. 14.

### DETAILED DESCRIPTION OF PREFERRED EMBODIMENTS

Preferred embodiments of the present invention will now be described in detail.

Embodiments of the present invention preferably include one or more systems and methods for whole-genome replacement in accordance with one or more processes that are sometimes referred to herein, not by way of limitation, but rather for purposes of convenience, as massively multiplexed homologous template repair (“MMHTR”).

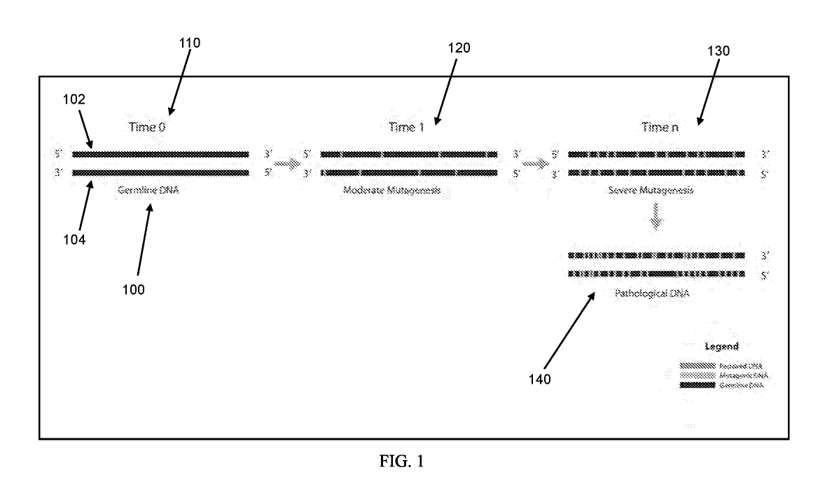

Referring now to FIG. 1, FIG. 1 illustrates a natural accumulation of genetic mutations in the nuclear DNA of a cell over time. It is known that during the course of a cell's life, the cell's DNA becomes mutated due to many factors and agents. Most of the mutations are corrected by the cell's DNA repair mechanisms. However, the repair mechanisms are imperfect and therefore some mutations remain uncorrected. Over time, the number of uncorrected mutations increases and eventually such mutations collectively cause the DNA sequence to be substantially different than the initial (e.g., germline) DNA sequence. Depending on the number of mutations in the DNA sequence and the location of the mutations in the DNA sequence, such differences can lead to many pathologies, including but not limited to various types of cancer, neurodevelopmental disorders (such as, for example, lissencephaly and hemimegalencephaly), development disorders (such as, for example, Proteus syndrome, McCune-Albright syndrome, Maffucci syndrome, Sturge Weber syndrome, and Beckwith-Wiedemann Syndrome), and other disorders (such as, for example, chronic autoimmune disease, neurofibromatosis, paroxysmal nocturnal hemoglobinuria, and incontinentia pigmenti).

The DNA 100 illustrated by FIG. 1 is represented in FIG. 1 in a standard manner known in the art, with an upper bar 102 representing a molecule strand having a direction, denoted as being from 5′ (read as “5 prime”) to 3′ (read as “3 prime”), and a lower bar 104 representing an opposing molecule strand having an opposite direction, denoted as being from 3′ to 5′, to show that the DNA molecules in the strand represented by the upper bar are complementary to the corresponding DNA molecules in the strand represented by the lower bar. As known in the art, the opposing molecules of the opposing strands are bound to one another such that the strands together form a double-helix structure. Black areas of each bar represent non-mutated portions of the molecular sequence. Green areas of each bar represent mutated portions of the molecular sequence.

When discussed herein, depicted DNA when referenced will include the two bound molecular strands thereof, and each depicted molecular strand when referenced will be referenced as either an “upper strand” or a “lower strand” not for purposes of limitation but for purposes of convenience in distinguishing one strand versus the other in the applicable figure, based on their relative illustrated positions in the figure.

More specifically, as illustrated in FIG. 1, the DNA 100 initially (e.g., at Time 0) is in a germline state, meaning, for example, a state in which no mutations are present (see, e.g., reference 110). Over time (e.g., during a time period starting at Time 0 and ending at Time 1), mutations occur and the DNA becomes moderately mutagenic, such that mutations are present but have little effect on the well-being of the individual (see, e.g., reference 120). As more time passes (e.g., during a time period starting at Time 1 and ending at Time n), mutations continue to occur and the DNA becomes severely mutagenic, such that a greater number of mutations are present but still have little effect on the well-being of the individual (see, e.g., reference 130), except that the likelihood increases that the DNA will become pathological. As still more time passes (e.g., during a time period starting at Time n and ending later), depending on many factors, the DNA becomes pathologically mutagenic (see, e.g., reference 140), such that the well-being of the individual is negatively affected. Primary examples of pathological DNA include, but are not limited to, cancer, neurodevelopmental disorders (such as, for example, lissencephaly and hemimegalencephaly), development disorders (such as, for example, Proteus syndrome, McCune-Albright syndrome, Maffucci syndrome, Sturge Weber syndrome, and Beckwith-Wiedemann Syndrome), and other disorders (such as, for example, chronic autoimmune disease, neurofibromatosis, paroxysmal nocturnal hemoglobinuria, and incontinentia pigmenti).

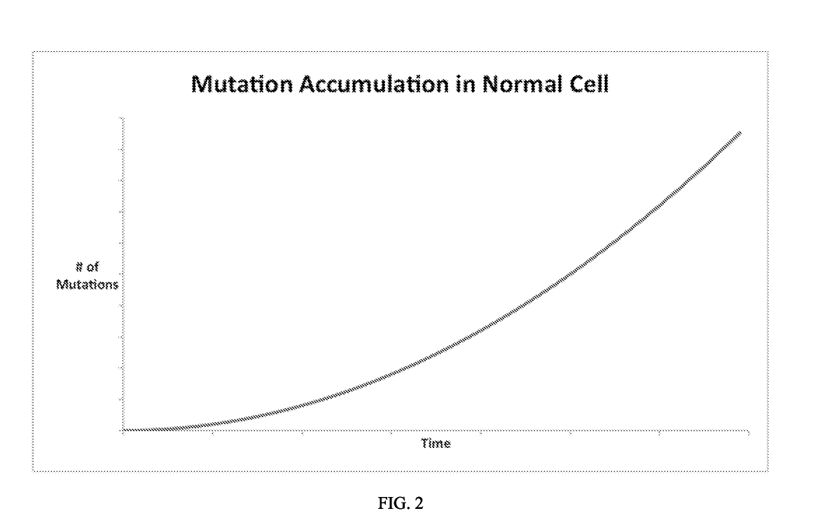

Referring now to FIG. 2, FIG. 2 illustrates an example of the total accumulation of mutations in a cell over time.

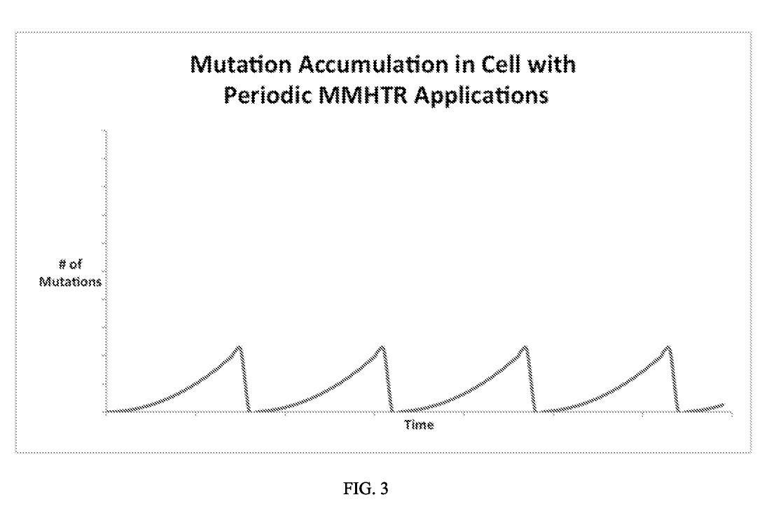

Referring now to FIG. 3, FIG. 3 illustrates an example of results of repeated applications of change agent material of an embodiment of the present invention, to periodically decrease, back to germline amounts, the number of mutations in a cell.

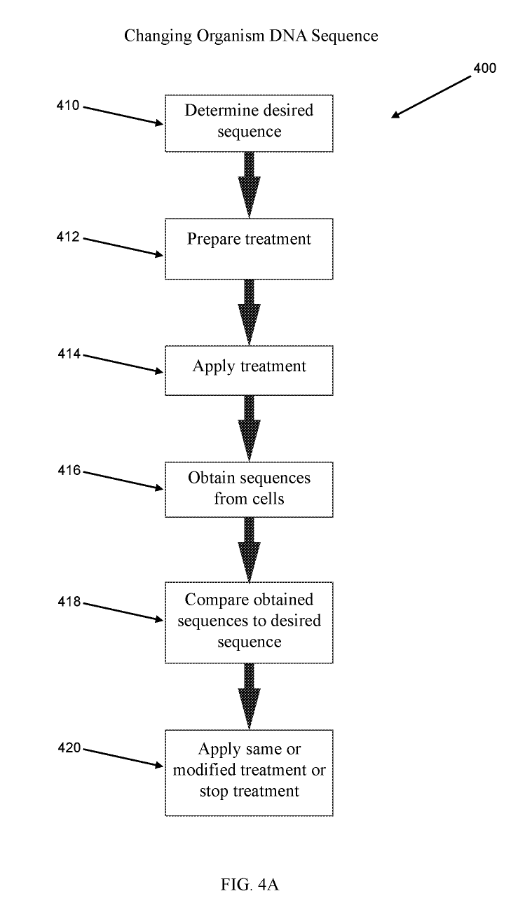

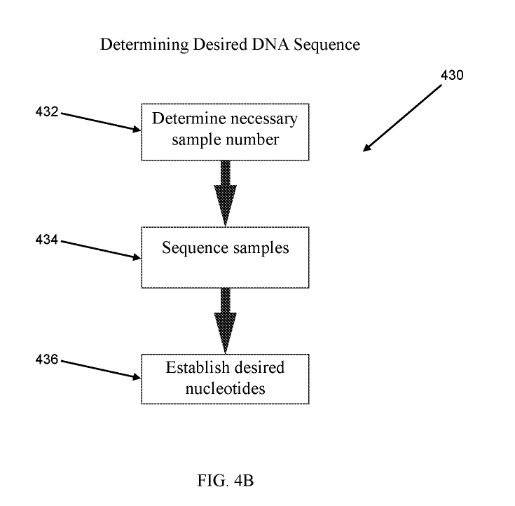

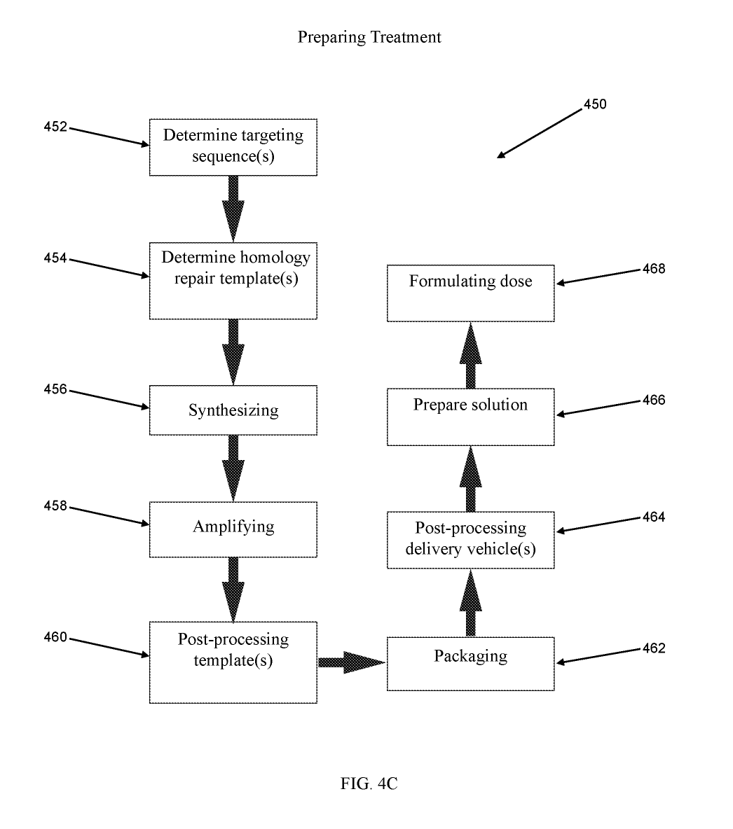

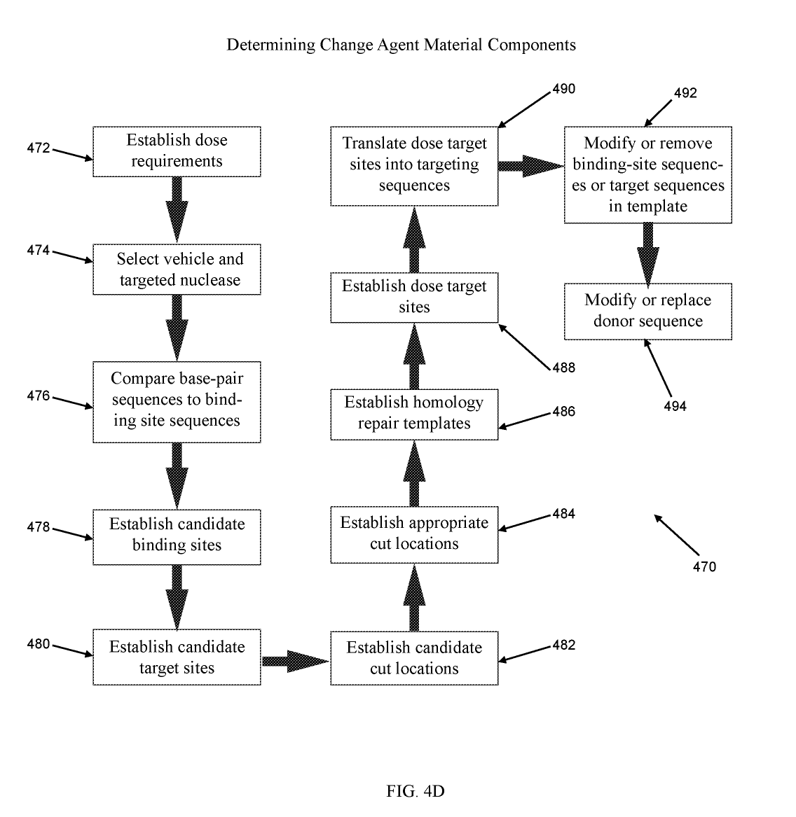

FIG. 4A illustrates a preferred method of an embodiment of the present invention, for changing an organism's DNA sequence. This process will be discussed in greater detail below.

FIG. 4B illustrates a preferred method of an embodiment of the present invention, for determining a desired DNA sequence of an embodiment of the present invention. This process will be discussed in greater detail below.

FIG. 4C illustrates a preferred method of an embodiment of the present invention, for preparing a treatment of an embodiment of the present invention. This process will be discussed in greater detail below.

FIG. 4D illustrates a preferred method of an embodiment of the present invention, for determining change agent material of an embodiment of the present invention. This process will be discussed in greater detail below.

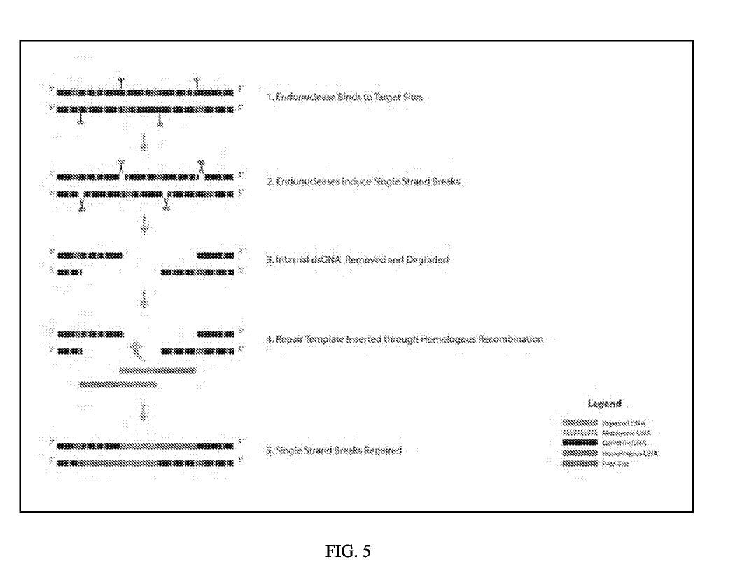

FIG. 5 illustrates, as to an embodiment of the present invention, the effect of applying, to a mutagenic DNA site, change agent material of an embodiment of the present invention, in accordance with an embodiment of the present invention. This effect will be discussed in greater detail below.

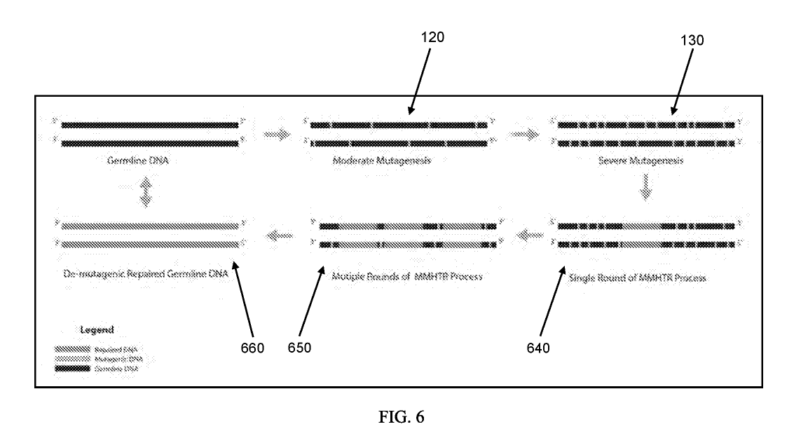

FIG. 6 illustrates the reversal of the accumulation mutagenic DNA through use of an embodiment of the present invention, such as, for example, a MMHTR process of the present invention. More specifically, as illustrated in FIG. 6, a preferred MMHTR process of the present invention is preferably applied (see, e.g., references 640 and 650) to cells having DNA in a state of severe mutagenesis (see, e.g., reference 130), although it should be understood that a preferred MMHTR process can be applied to cells having DNA in any state of mutagenesis, such as, for example without limitation, a state of moderate mutagenesis (see, e.g., reference 120), a state of severe mutagenesis (see, e.g., reference 130), and/or a state of pathological mutagenesis (see, e.g., reference 140 on FIG. 1).

While a preferred MMHTR process can be applied to cells having DNA in any state of mutagenesis, the effectiveness of the MMHTR process on cells with DNA in a state of greater mutagenesis may be less than the effectiveness of the MMHTR process on cells with DNA in a state of lesser mutagenesis, depending on the following factors: the amount of mutation in the target sites and how different the target sites are from the targeting sequences generated to bind to them; the amount of mutation in a sequence of the DNA that is homologous to the homology arm sequence, and how different it is from the homology arm sequence generated to bind to it; and mutations affecting the cells intrinsic repair mechanism's efficiency and function (e.g., the more mutations, the greater the possibility that the cell cannot effectuate homologous recombination effectively enough to repair the DNA through a MMHTR process).

Similarly, while a preferred MMHTR process can be applied to cells having DNA in any state of mutagenesis, the speed of the effectiveness of the MMHTR process on cells with DNA in a state of greater mutagenesis may be less than the speed of the effectiveness of the MMHTR process on cells with DNA in a state of lesser mutagenesis, depending on the following factors: mutations affecting the cell's intrinsic repair mechanism's efficiency and function (e.g., the more mutations, the greater the possibility that the cell cannot effectuate homologous recombination effectively enough to repair the DNA through a MMHTR process); and mutations affecting the ability of the cell to transcribe the injected mRNA or plasmid of the vehicle payload, if that is the method through which the payload is delivered.

However, the present invention is not limited to the application of a MMHTR process on cells with any particular degree of mutagenesis, but instead encompasses the application of a MMHTR process on cells with any degree, and all degrees, of mutagenesis.

Further, as illustrated in FIG. 6, a single round (e.g., a dose including change agent material) of a preferred MMHTR process can be applied (see, e.g., reference 640), but preferably multiple rounds (multiple doses, each having respective change agent material) of a preferred MMHTR process are applied (see, e.g., reference 650). (It should be understood that each round of a MMHTR process, rather than being configured to change only a single site of the organism DNA sequence to be changed, is configured to change multiple sites of the organism DNA sequence to be changed.) Such multiple rounds can be applied serially or applied in parallel, or in various combinations of serial and parallel applications. For example, without limitation, the present invention encompasses serial applications of multiple MMHTR rounds, each of which includes parallel applications of multiple MMHTR rounds. Also, for example, without limitation, the present invention encompasses parallel applications of multiple MMHTR rounds, each of which includes serial applications of multiple MMHTR rounds.

Further as illustrated in FIG. 6, one or more rounds of the application of the MMHTR process, on a cell having mutagenic DNA, preferably results in the cell having DNA that is in a state identical to the DNA's initial germline state (see, e.g., reference 660), due to the de-mutagenic repair of the DNA effected by the one or more applied MMHTR processes. It should be understood that each blue line opposing pair depicted in FIG. 6 indicates multiple separate areas of a DNA sequence being changed by a single round of the MMHTR process.

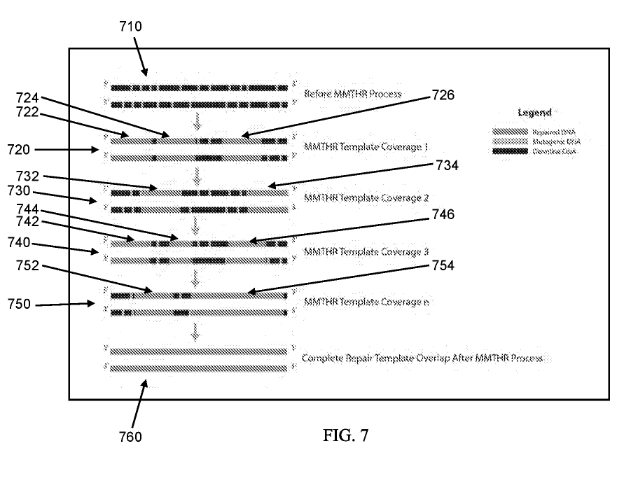

FIG. 7 illustrates the application of multiple rounds of the MMHTR process of the present invention on a cell having mutagenic DNA. It is illustrated that prior to application of the MMHTR rounds, the DNA is in a state of mutagenesis (see, e.g., reference 710). Rounds of MMHTR processes can be applied in series (see, e.g., references 720, 730, 740 and 750), in which each set of serial rounds apply MMHTR rounds in parallel (see, e.g., references 722, 724, and 726 applied in parallel; 732 and 734 applied in parallel but in sequence after the rounds illustrated by reference 720; 742, 744 and 746 applied in parallel but in sequence after the rounds illustrated by reference 730; and 752 and 754 applied in parallel but in sequence after the rounds illustrated by reference 740), until the combined coverage of the MMHTR rounds results in a complete return of the DNA to a non-mutagenic state (see, e.g., reference 760). It should be understood that each blue line opposing pair depicted in FIG. 7 indicates multiple separate areas of a DNA sequence being changed by a single round of the MMHTR process.

It should be understood that while FIG. 7 illustrates multiple rounds of MMHTR processes applied in series, in which each of the multiple rounds includes multiple rounds of MMHTR processes applied in parallel, the scope of the present invention encompasses single MMHTR processes being applied, and also encompasses multiple MMHTR processes being applied, in any effective timing and order, whether serially or in parallel, or combinations of both serial and parallel applications. Further, the scope of the present invention encompasses multiple MMHTR processes being applied in a predetermined order, or in no predetermined order, such that, for example without limitation, in some embodiments, the MMHTR processes are purposefully initiated but progress and complete according to a timing determined by the variable proximity of the targeted sites to the change agent material, and/or by other factors.

Referring again to FIG. 5, the effect, on a single mutagenic DNA site, of a MMHTR round will be described in greater detail. By way of explanation but not to limit the scope of the present invention, each single MMHTR round (e.g., instance of delivery of change agent material) targets at least two sites in the mutagenic DNA, and multiple processes applied to multiple sites can collectively affect the entire DNA sequence. FIG. 5 illustrates an effect of a single MMHTR round on a single one of the multiple mutagenic DNA sites affected by the MMHTR round. It should be understood that each blue line opposing pair depicted in FIG. 5 indicates a single site of a DNA sequence being changed, as one of the multiple mutagenic DNA sites affected by a single MMHTR round.

As to each MMHTR process, FIG. 5 illustrates one example of the effect, on the mutagenic DNA, of the MMHTR process. Preferably, upon administration of the prepared solution, first the endonucleases bind to the target sites. (It should be understood that the mechanism of target site detection and binding is dependent on the type of endonuclease used.)

Preferably, second, the endonucleases induce single strand breaks. The illustrated example assumes the use of an endonuclease that achieves single strand cuts (e.g., nicks) on the targeted DNA molecule. (It should be understood that the type, location and mechanism of cutting is dependent on the type of endonuclease used.)

Preferably, third, the internal dsDNA is removed and degraded. While the mechanism of removal and degradation is not illustrated, the result of removal is shown. (It should be understood that the mechanism of homologous recombination in the cell is a combination of a variety of steps by which the homologous repair template binds to the targeted DNA strand, the cut DNA is displaced and removed, and the cuts in the targeted DNA strand are repaired (e.g., ligased).)

Preferably, fourth, the repair template is inserted through homologous recombination. (It should be understood that the illustrated example does not show the steps required for homologous recombination, nor does it show alternative repair pathways that could occur that would not result in homologous recombination. The illustrated example shows the intended end result of a successful homologous recombination event.)

Preferably, fifth, the single strand breaks are repaired. The illustrated example shows the repairing of single strand nicks in the targeted DNA strand through the action of an intrinsic ligase (e.g., DNA ligase repairs the connection between DNA base pairs in a single strand).

Preferred Method of Substantially Changing a DNA Sequence of an Organism

Referring again to FIG. 4A, in a preferred embodiment, a method of the present invention includes a method of substantially changing a DNA sequence of an organism (see, e.g., reference 400), the method including but not limited to one or more of the following steps:

determining a desired DNA sequence (see, e.g., reference 410), the desired DNA sequence being a DNA sequence to which it is desired that the DNA sequence of the organism be substantially changed;

preparing a treatment (see, e.g., reference 412) configured to cause the organism DNA sequence to be substantially changed to the desired DNA sequence;

applying the treatment (see, e.g., reference 414) to the organism;

wherein

the treatment is configured to cause the organism DNA sequence to be substantially changed to the desired DNA sequence by causing, at each of multiple sites in the organism DNA sequence, genetic code at the site to be substantially changed to genetic code at a corresponding site in the desired DNA sequence;

each of the multiple sites in the organism DNA sequence is a respective sub-sequence of the organism DNA sequence;

applying the treatment includes delivering to the organism at least one dose; and

each dose includes respective change agent material that causes the changing of the genetic code at a respective plurality of the multiple sites.

Preferably, the DNA sequence of the organism is a whole-genome DNA sequence of the organism. However, it should be understood that in other embodiments, the DNA sequence of the organism can be a DNA sequence that is less than a whole-genome DNA sequence, and rather a sub-sequence of a whole-genome DNA sequence. It should be further understood that the system and method of the present invention also can be used to substantially change one or more other types of nucleic acid sequences, of any possible length.

Preferred Desired DNA Sequences

Preferably, the desired DNA sequence is substantially at least one of a germline whole-genome DNA sequence of the organism, a pre-mutagenic whole-genome DNA sequence of the organism, a global average whole-genome DNA sequence of the organism, a whole-genome DNA sequence of another organism, and a whole-genome DNA sequence that is an intentionally modified version of a germline whole-genome DNA sequence of the organism.

More specifically, without limiting its meaning in the art, a germline whole-genome DNA sequence of an organism can be understood herein to mean the DNA sequence that represents the source of DNA for all cells of the organism.

Further specifically, without limiting its meaning in the art, a pre-mutagenic whole-genome DNA sequence of an organism is understood herein to mean the entire genomic DNA sequence without any mutations acquired after conception, or the entire genomic DNA sequence without any inherited mutations.

Further specifically, without limiting its meaning in the art, a global average whole-genome DNA sequence of an organism is understood herein to mean the entire genomic DNA sequence of the organism, where each nucleotide letter in a position in the sequence is the most common nucleotide letter at that position in a statistically relevant sample of sequences.

Further specifically, without limiting its meaning in the art, an organism can be a human, a non-human animal, a plant, and/or any entity having a DNA sequence.

As to the above indication that the desired DNA sequence can be a whole-genome DNA sequence of another organism, it should be understood that it is contemplated by the present invention that the present invention can be used to change the DNA of an organism into the DNA of another organism (whether or not the same type of organism). Therefore, in the case of whole-genome replacement using the present invention, it should be understood that it is contemplated that the whole-genome DNA sequence of an organism can be, using the present invention, changed into the whole-genome DNA sequence of another organism, effectively changing the one organism into the other, genetically speaking not by way of limitation.

As to the above indication that the desired DNA sequence can be a whole-genome DNA sequence that is an intentionally modified version of a germline whole-genome DNA sequence of the organism, it should be understood that intentional modifications to an organism's DNA can be effected using the present invention. Purposes of such modifications can include, but are not limited to, additions, eliminations, enhancements, or reductions of existing features and/or abilities, and additions and/or eliminations of disorders or diseases. More specifically, the present invention can be used to replace a whole-genome DNA sequence of an organism with a whole-genome DNA sequence that is substantially similar in all respects to the whole-genome DNA sequence being replaced except for intentional modifications that will effect the desired outcome as to features, abilities, disorders and/or diseases. For example, without limitation, modifications can be made to effect desired changes in aesthetics (e.g., changes to an organism's appearance, such as, for example, eye color), dimensions (e.g., changes to an organism's physical traits such as, for example, height), processes (e.g., changes to an organism's biological processes, such as, for example, metabolic function), and abilities (e.g., changes to an organism's capabilities, such as, for example, sharper eyesight). For example, without limitation, modifications can be made to correct or prevent disorders or cure or prevent diseases, such as, for example, cancer, neurodevelopmental disorders (such as, for example, lissencephaly and hemimegalencephaly), development disorders (such as, for example, Proteus syndrome, McCune-Albright syndrome, Maffucci syndrome, Sturge Weber syndrome, and Beckwith-Wiedemann Syndrome), and other disorders (such as, for example, chronic autoimmune disease, neurofibromatosis, paroxysmal nocturnal hemoglobinuria, and incontinentia pigmenti).

It may be difficult to determine an organism's germline whole-DNA sequence, due to inefficiencies in sequencing technology and/or an inability to know for certain which cell, if any, of the organism contains a whole-genome DNA sequence that is not at all mutated from its original germline state. In such cases, various technologies can be used to obtain a whole-genome DNA sequence that is as accurate a representation of the organism's germline whole-genome DNA sequence as possible. Such technologies at present include, but are not limited to, chain-termination sequencing (Sanger Sequencing), Maxam-Gilbert sequencing, shotgun sequencing, single-molecule real-time sequencing, pyrosequencing, sequencing by synthesis, sequencing by ligation (SOLiD sequencing), nanopore sequencing, and massively parallel signature sequencing. However, it should be understood that any method that is now known or will be developed or otherwise known in the future, is contemplated for use in the present invention to determine as accurate a representation of the organism's germline whole-genome DNA sequence as possible. Further, if a method presently exists, or exists in the future, to determine with certainty an organism's germline whole-genome DNA sequence, it is contemplated that such a method can be used in the present invention to determine an organism's germline whole-genome DNA sequence, such that the same can be used as the desired DNA sequence in the method of the present invention.

Preferred Additional or Alternate Method of Determining a Desired DNA Sequence

As an additional or alternate method of determining a desired whole-genome DNA sequence (see, e.g., reference 430), in certain embodiments of the present invention, the desired DNA sequence is a desired whole-genome DNA sequence, the desired sequence having a plurality of nucleotides each at a respective nucleotide position in the desired sequence, the desired sequence being determined by a method including but not limited to one or more of the following steps:

determining a necessary sample number (see, e.g., reference 432), the necessary sample number being equal to a number of whole-genome DNA sequence samples, each having a plurality of nucleotides each at a respective nucleotide position in the sample, each sample being from a different cell of the organism, that must be sequenced to establish that, for each of the nucleotide positions in the desired sequence, the nucleotide at the corresponding nucleotide position in a majority of the samples is the nucleotide desireable for the nucleotide position in the desired sequence;

sequencing (see, e.g., reference 434) the necessary sample number of whole-genome DNA sequence samples to determine, for each of the samples, a respective sample sequence; and

establishing (see, e.g., reference 436) that, for each nucleotide position in the desired sequence, the nucleotide at the nucleotide position in the desired sequence is the nucleotide at the corresponding nucleotide position in a majority of the samples.

Preferably as to this method of determining a desired whole-genome DNA sequence, in certain embodiments of the present invention, a germline whole-genome DNA sequence of the organism has a plurality of nucleotides each at a respective nucleotide position in the germline sequence, and the nucleotide desireable for the nucleotide position in the desired sequence is the nucleotide in the corresponding nucleotide position in the germline sequence.

Further preferably as to this method of determining a desired whole-genome DNA sequence, in certain embodiments of the present invention, the necessary sample number is equal to a number of the whole-genome DNA sequence samples that must be sequenced to establish that a probability, that for each sample, each nucleotide in its respective nucleotide position in the sample is not the nucleotide at the corresponding nucleotide position in the germline sequence, is less than a desired probability, the desired probability being no greater than one divided by the total number of nucleotides in the germline sequence.

Further preferably as to this method of determining a desired whole-genome DNA sequence, in certain embodiments of the present invention, the desired probability is a product of a first factor and a second factor, the first factor being an error rate of a sequencing method used to sequence at least one of the samples, the second factor being a number no greater than one divided by the total number of nucleotides in the germline sequence.

Preferably as to this method of determining a desired whole-genome DNA sequence, in certain embodiments of the present invention, determining the necessary sample number includes accounting for an error rate of a sequencing method used to sequence at least one of the samples.

Preferred Application of Treatment

Preferably, as to the above described method of substantially changing a DNA sequence of an organism, applying the treatment preferably includes administering an aqueous solution to one or more cells of the organism. More specifically, as will be discussed in greater detail below, certain preferred embodiments of the present invention result in the treatment taking the form of an aqueous solution, and in some of such embodiments, it is preferable to administer the aqueous solution to one or more cells of the organism.

Preferably, administering the solution includes at least one of injecting the solution into the organism adjacent to the one or more cells and intravenously introducing the solution into a bloodstream of the organism.

In certain embodiments, injecting the solution into the organism adjacent to the one or more cells can be achieved by hypodermic needle injection or any effective method now known or later developed method.

In certain embodiments, intravenously introducing the solution into a bloodstream of the organism can be achieved by any effective method now known or later developed.

Preferred Comparison of Organism DNA to Desired DNA Sequence

Referring again to FIG. 4A, preferably, as to the above described method of substantially changing a DNA sequence of an organism, the method further includes but is not limited to one or more of the following steps:

obtaining (see, e.g., reference 416), from each of a plurality of cells of the organism, the cells being randomly chosen from among cells of interest, a DNA sequence from the cell;

comparing (see, e.g., reference 418) each of the obtained DNA sequences to the desired DNA sequence; and

based on results of the comparison, taking one or more of the following steps (see, e.g., reference 420): applying the treatment to the organism again, applying to the organism a treatment modified based on results of the comparison, and determining that no additional treatment applications are necessary.

Preferably, such comparisons are useful to, for example, determine the amount and type of modifications that have occurred in the organism's cells as a result of the treatment. Understanding the differences between the DNA sequences of the cells, the sequences' original state, and the sequences' intended state (e.g., the state expected as a result of the treatment) helps determine the modifications that occurred and what, if any, are still required or desired.

Preferred Change Agent Material

Preferably, as to the above described method of substantially changing a DNA sequence of an organism, the change agent material is useful to treat the organism but not useful to treat any other organism.

More specifically, for example without limitation, in contrast to prior methods of modifying DNA sequences using CRISPR, which are all directed to affecting small sub-sequences of DNA that are known to relate to a disorder or disease in a similar way for most members of a species of organism, and as such can effectively be applied to treat multiple members of the species, the whole genome replacement process of the present invention is preferably highly customized to an individual member of a species, such that a treatment of the present invention would be effective only for an organism with a specific whole-genome DNA sequence.

However, in some cases, members of a species of organism share the same germline whole-genome DNA sequence, such as, for example, without limitation, identical twins, triplets, quadruplets, n-tuplets, etc. Therefore, in those and other cases, it is contemplated that the present invention can be used to prepare one or more treatments in which one or more doses (e.g., rounds) and/or the change agent material of such one or more doses (e.g., rounds) is useful to treat more than one organism.

Further, the present invention can be used to affect one or more sub-sections of a whole-genome DNA sequence, rather than to affect an entire whole-genome DNA sequence. Therefore, in certain cases, it is contemplated that the present invention can be used to prepare one or more treatments in which one or more doses (e.g., rounds) and/or the change agent material of such one or more doses (e.g., rounds) is useful to treat two or more organisms that do not have the same germline DNA. More specifically, for organisms that do not have the same germline DNA but who have one or more sub-sequences of their germline DNA that are the same, the present invention can be used to prepare a treatment such that when the same treatment is applied to the organisms, such sub-sequences in the organisms are changed. For example, without limitation, certain types of cancer, for which the sequences of the cancer mutations are known, can be targeted in the same manner for different people using the present invention.

Preferred Preparation of Treatment

Referring now to FIG. 4C, preferably, as to the above described method of substantially changing a DNA sequence of an organism, preparing the treatment (see, e.g., reference 450) preferably includes but is not limited to one or more of the following steps:

determining, for each of the multiple sites, at least one targeting sequence (see, e.g., reference 452);

determining, for each of the multiple sites, at least one homology repair template based on at least one characteristic of the at least one targeting sequence for the site (see, e.g., reference 454);

synthesizing the targeting sequences and the homology repair templates (see, e.g., reference 456);

amplifying the targeting sequences and the homology repair templates (see, e.g., reference 458);

post-processing the homology repair templates (see, e.g., reference 460);

packaging the targeting sequences and the homology repair templates into at least one delivery vehicle (see, e.g., reference 462);

post-processing the delivery vehicle (see, e.g., reference 464), wherein post-processing the delivery vehicle includes at least one of increasing delivery success and decreasing side effects;

preparing a solution that includes the change agent material (see, e.g., reference 466), the change agent material including the post-processed delivery vehicle including the targeting sequences, the post-processed homology repair templates, and at least one of at least one targeted nuclease protein and at least one nucleic acid sequence that expresses at least one targeted nuclease protein; and

formulating the dose (see, e.g., reference 468), the dose including the solution; wherein formulating the dose includes modifying, for treating the organism, the solution as to one or more of the following aspects: concentration, molarity, dosage and content.

Preferably, the post-processing of the homology repair template(s) includes post-processing to avoid and/or minimize any adverse epigenetic effects. Such post-processing can include, for example without limitation, methylation.

Preferred Determination of Change Agent Material Components

Referring now to FIG. 4D, preferably, the change agent material includes a plurality of targeting sequences, a plurality of homologous repair templates based on at least one of the plurality of targeting sequences, and at least one of at least one targeted nuclease protein and at least one nucleic acid sequence that expresses at least one targeted nuclease protein.

Further preferably, as to the above described method of preparing the treatment, preferably the targeting sequences and homology repair templates are determined by, the delivery vehicle is chosen by, and at least one of the at least one targeted nuclease protein and the at least one nucleic acid sequence are chosen by, repeating a process (see, e.g., reference 470) until all desired changes to the organism DNA sequence are expected to result from use of the change agent material in the dose, the process including but not limited to one or more of the following steps:

for the dose, establish at least one dose requirement (see, e.g., reference 472), the at least one dose requirement being based on at least one dose criterion, the at least one dose criterion being one or more of at least one delivery vehicle characteristic, at least one targeted nuclease characteristic, at least one off-target effect characteristic, at least one cutting mechanism characteristic, at least one organism characteristic, and at least one targeting sequence characteristic;

select a delivery vehicle and a targeted nuclease that meet the at least one dose requirement to a desired degree (see, e.g., reference 474);

for each of a desired number of base pair sequences of the organism DNA sequence, when the targeted nuclease requires at least one binding site sequence specific to the targeted nuclease, compare the base pair sequence to the at least one binding site sequence (see, e.g., reference 476);

for each of the compared base pair sequences, when the base pair sequence is compatible with the specific at least one binding site sequence, establish as a respective candidate binding site (see, e.g., reference 478) a binding site defined by binding site location information of the base pair sequence, the binding site location information including a start location, an end location, and a base pair letter sequence;

for each candidate binding site, using target site selection rules specific to the targeted nuclease, establish as a respective candidate target site (see, e.g., reference 480) a target site defined by target site location information associated with the candidate binding site, the target site location information including a start location and an end location, the rules including one or more of a distance from the candidate binding site and a base pair length;

for each candidate target site, using a cutting profile of the targeted nuclease, establish as a respective candidate cut location (see, e.g., reference 482) a location associated with the candidate target site and at which a cut is most likely to occur;

for each candidate cut location, establish the candidate cut location as a respective appropriate cut location (see, e.g., reference 484) based on at least one cut location suitability factor, the at least one cut location suitability factor being one or more of a homology repair template maximum size, a homology repair template homology arm size, and a donor sequence maximum size;

establish a plurality of homology repair templates (see, e.g., reference 486) by, for each homology repair template, determining a homology repair template target group, the group including at least two cut locations of the appropriate cut locations, the group meeting at least one compatibility requirement with respect to at least one other homology repair template target group;

for each appropriate cut location, establish as a respective dose target site (see, e.g., reference 488) the candidate target site associated with the appropriate cut location; and

translate each dose target site into a respective targeted nuclease targeting sequence (see, e.g., reference 490).

Preferred Modification of Repair Template(s)

Preferably, the process further includes but is not limited to, for at least one homology repair template, mutating or removing any targeted nuclease binding site sequences or donor sequences in the homology repair template (see, e.g., reference 492).

For example, it may be desirable to avoid or reduce the possibility that the target nuclease would bind to and cut at a binding site sequence (e.g., a PAM site sequence). For example, if the binding site sequence is removed from the template, such a possibility could be avoided or reduced.

Preferred Modification of Donor Sequence(s)

Preferably, the process further includes but is not limited to, for at least one homology repair template, modifying or replacing a donor sequence to include the desired DNA sequence (see, e.g., reference 494).

For example, it may be desireable to, after determining one or more homology repair templates, to “swap out” one or more donor sequences in the templates for different donor sequences, for the purpose of, without limitation, effecting changes different than changes originally intended and/or different than changes that would have been caused by the original donor sequences.

Preferred Dose Criteria

Preferably, the at least one delivery vehicle characteristic is selected from the group consisting of a delivery vehicle size, a delivery vehicle cost, a delivery vehicle ease of ingestion, a delivery vehicle successful payload delivery likelihood, a delivery vehicle delivery route, and a delivery vehicle immunogenicity;

the at least one targeted nuclease characteristic is selected from the group consisting of a targeted nuclease targeting specificity, a targeted nuclease targeting consistency, a targeted nuclease binding specificity, a targeted nuclease binding consistency, a targeted nuclease cutting specificity, a targeted nuclease cutting consistency, targeted nuclease cost, a targeted nuclease required binding sequence distribution, a targeted nuclease required binding sequence probability, a targeted nuclease required binding sequence quantity;

the at least one off-target effect characteristic is selected from the group consisting of a likelihood of off-target effect occurrence, a likelihood of off-target effect amounts, and a likelihood of off-target cuts;

the at least one cutting mechanism characteristic is selected from the group consisting of a cutting mechanism type and a cutting mechanism action;

the at least one organism characteristic is selected from the group consisting of an organism nuclease activity amount and an organism nuclease degradation rate; and

the at least one targeting sequence characteristic is a targeting sequence length.

Preferred Compatibility Requirements

Preferably, the at least one compatibility requirement is selected from the group consisting of a homology repair template maximum size, a donor sequence overlap limit, a homology repair template overlap limit, a number of cuts to be made in the organism DNA sequence; a presence of a targeted nuclease binding site within a homology repair template; and a presence of a donor sequence associated with a targeted nuclease binding site within a homology repair template.

Example with CRISPR-Cas9 Selected as Targeted Nuclease

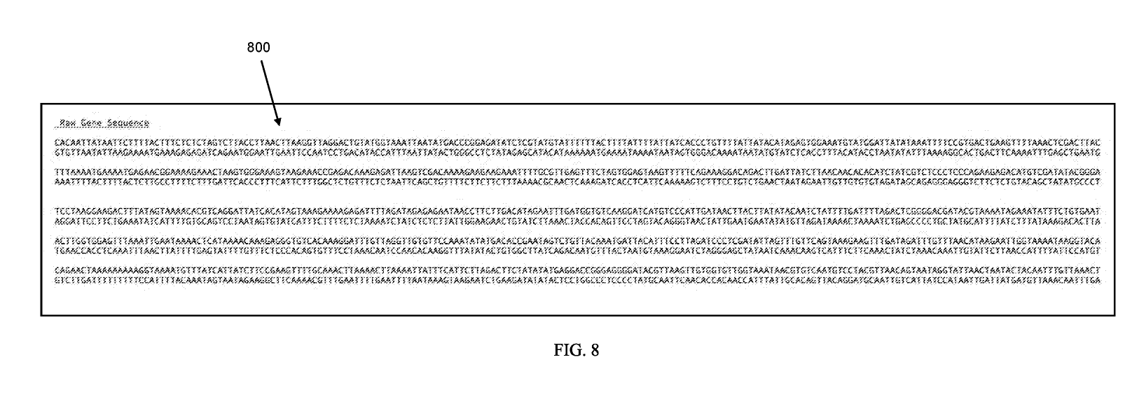

Referring now to FIGS. 8-16 and with reference again to FIGS. 4C and 4D, an example of determining change agent material components in accordance with an embodiment of the present invention will be discussed.

As discussed above, with regard to preparing a treatment in accordance with an embodiment of the present invention, preferably the targeting sequences and homology repair templates are determined by, the delivery vehicle is chosen by, and at least one of the at least one targeted nuclease protein and the at least one nucleic acid sequence are chosen by, repeating a process (see, e.g., reference 470) until all desired changes to the organism DNA sequence are expected to result from use of the change agent material in the dose, the process preferably including one or more of the indicated steps.

Example of Establishing Dose Requirements

Also as discussed above, an initial step is to, for the dose, establish at least one dose requirement, the at least one dose requirement being based on at least one dose criterion (see, e.g., reference 472). Preferably, in this step, dose criterion can include, but are not limited to, the following: how specifically and consistently a targeted nuclease targets, binds and cuts at a desired target site; presence and amount of likely off-target effects, including but not limited to off-target cuts; targeted nuclease targeting sequence length; distribution, probability and quantity of targeted nuclease associated required binding site sequences in the targeted DNA strand; cut type and action, including double strand breaks, single strand breaks (e.g., nicks) and others; amount of nuclease activity and degradation time in organism; cost of targeted nuclease; size of delivery vehicle; cost of delivery vehicle; ease of ingestion of delivery vehicle; likelihood of successful payload (e.g., change agent material) delivery of delivery vehicle; delivery route of delivery vehicle; and immunogenicity of delivery vehicle.

### Example

In the example, the following dose criteria were considered relevant: cut type, and size of delivery vehicle. As to the cut type, a single strand break (e.g., nick) was considered preferable, because, for example, single strand break is understood to increase the specificity of the cut. As to the size of the delivery vehicle, 4000-5000 base pairs (bp) was considered preferable, because, for example, a largest possible size available from known vehicles was preferred, and a vehicle meeting the size requirement had well understood properties, and human body response to the vehicle was well understood.

Example of Selecting Vehicle and Nuclease

Also as discussed above, a next step is to select a delivery vehicle and a targeted nuclease that meet the at least one dose requirement to a desired degree (see, e.g., reference 474).

### Example

In the example, it was determined that the following targeted nuclease and delivery vehicle met the selection criteria to a sufficient degree:
Cas9 Homologue: SpCas9
Cas9 Variant: SpCas9n
Sub Variant: D-10A
Cas9 size: 4101 bp
PAM sequence: 5′-NGG
sgRNA size: 131 bp
Delivery Vehicle: Adeno-associated Virus (AAV)
Delivery Vehicle Max Cargo Size: 4700 bp

### Example

For the example, the organism DNA sequence to which changes are to be applied has been identified and is shown in FIG. 8, and is referred to therein as the “Raw Gene Sequence” (see, e.g., reference 800), with letters representing molecules of the DNA strands (“A” indicating Adenine, “C” indicating Cytosine, “G” indicating Guanine, “T” indicating Thymine), adjacent one another in base pairs in a standard manner of DNA illustration known in the art.

Example of Comparing Base Pair Sequences to Binding Site Sequence

Also as discussed above, a next step is to, for each of a desired number of base pair sequences of the organism DNA sequence, when the targeted nuclease requires at least one binding site sequence specific to the targeted nuclease, compare the base pair sequence to the at least one binding site sequence (see, e.g., reference 476).

### Example

In the example, the selected targeted nuclease requires at least one binding site sequence specific to the targeted nuclease.

Preferably, in this step, the organism DNA sequence (preferably, the entire organism DNA sequence) is searched to find its base pair sequences, and the base pair sequences are found and read. Using the targeted nuclease binding site sequence (or sequences), for each base pair sequence read, the base pair sequence is compared to the targeted nuclease binding site sequence (or sequences).

### Example

In the example, the targeted nuclease is CRISPR-Cas9, which requires a PAM site sequence of “NGG” (e.g., where the “N” refers to any of the four types of nucleotides). Accordingly, each base pair sequence of the organism DNA sequence is read and compared to the PAM site sequence.

Example of Establishing Candidate Binding Sites

Also as discussed above, a next step is to, for each of the compared base pair sequences, when the base pair sequence is compatible with the specific at least one binding site sequence, establish as a respective candidate binding site (see, e.g., reference 478) a binding site defined by binding site location information of the base pair sequence, the binding site location information including a start location, an end location, and a base pair letter sequence.

Preferably, in this step, if the base pair sequence read during the search is equal to the targeted nuclease binding site sequence, then the start and end locations and base pair letters of the read base pair sequence (collectively, the binding site location information) are saved. Then, the targeted nuclease binding site location information is added to a list of candidate targeted nuclease binding sites for the organism DNA sequence.

Preferably, the process of searching the genome for the base pairs and finding them, comparing the base pairs to the binding site sequence, and adding matching locations to the list of candidate binding sites, continues until the entire organism DNA sequence is searched.

### Example

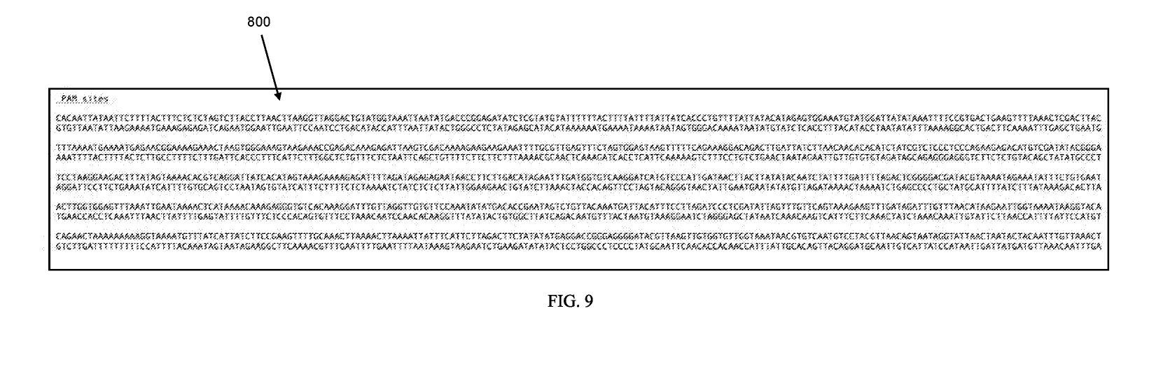

For the example, the candidate binding site locations of the organism DNA sequence (e.g., the PAM sites) are illustrated in FIG. 9 and indicated by yellow highlight.

Example of Establishing Candidate Target Sites

Also as discussed above, a next step is to, for each candidate binding site, using target site selection rules specific to the targeted nuclease, establish as a respective candidate target site (see, e.g., reference 480) a target site defined by target site location information associated with the candidate binding site, the target site location information including a start location and an end location, the rules including one or more of a distance from the candidate binding site and a base pair length.

Preferably, in this step, the rules (e.g., selection criteria) can include one or more of the following: distance of target site from targeted nuclease binding site, and total base pair length. Preferably, in this step, using those selection criteria, for each targeted nuclease binding site location in the list of candidate targeted nuclease binding sites, the start and end locations of the candidate target site associated to the found targeted nuclease binding site are calculated, and the target site and target site location information are added to a list of candidate target sites. Preferably, this step is repeated until the entire list of candidate targeted nuclease binding sites has been searched.

### Example

In the example, the selection criteria can include, but are not limited to, the distance of the target sequence from the Cas9-variant PAM site, and the total base pair length. Therefore, for each Cas9-variant PAM site location in the list of candidate Cas9-variant PAM sites, the start and end locations of the candidate target site associated to the found Cas9-variant PAM site are calculated. Then, the target site and target site location information is added to a list of candidate target sites.

### Example

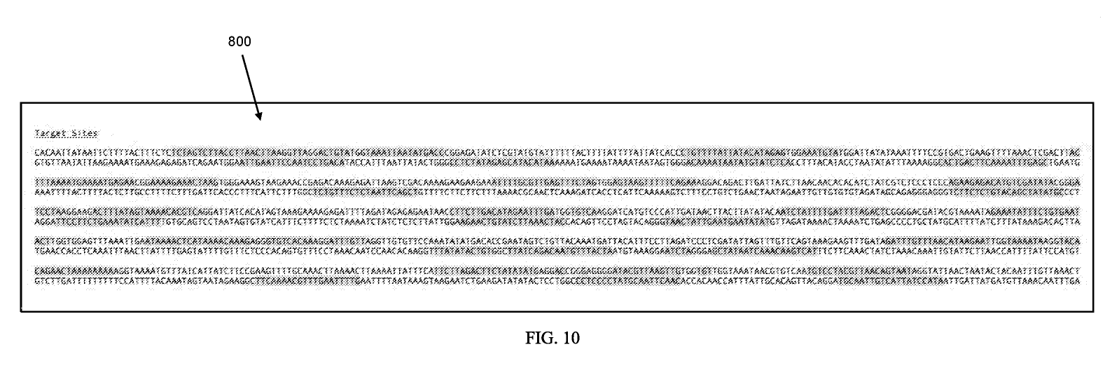

For the example, the candidate target sites of the organism DNA sequence are illustrated in FIG. 10 and indicated by green highlight. It should be noted that, as shown in the figure, it is possible that a binding site sequence (e.g., PAM site sequence) may reside in a candidate target site.

Example of Establishing Candidate Cut Locations

Also as discussed above, a next step is to, for each candidate target site, using a cutting profile of the targeted nuclease, establish as a respective candidate cut location (see, e.g., reference 482) a location associated with the candidate target site and at which a cut is most likely to occur.

Preferably, in this step, using the cutting profile of the selected targeted nuclease, for each target site in the list of candidate target sites, the action is to determine where the most likely cut will occur, and then add those candidate cut locations to a list of all candidate cut locations.

### Example

In the example, the targeted nuclease is CRISPR-Cas9, specifically CRISPR-Cas9 variant SpCas9n (D-10a), which has a cutting profile such that it cuts only in one DNA strand, and cuts the DNA strand opposite of where it lands.

### Example

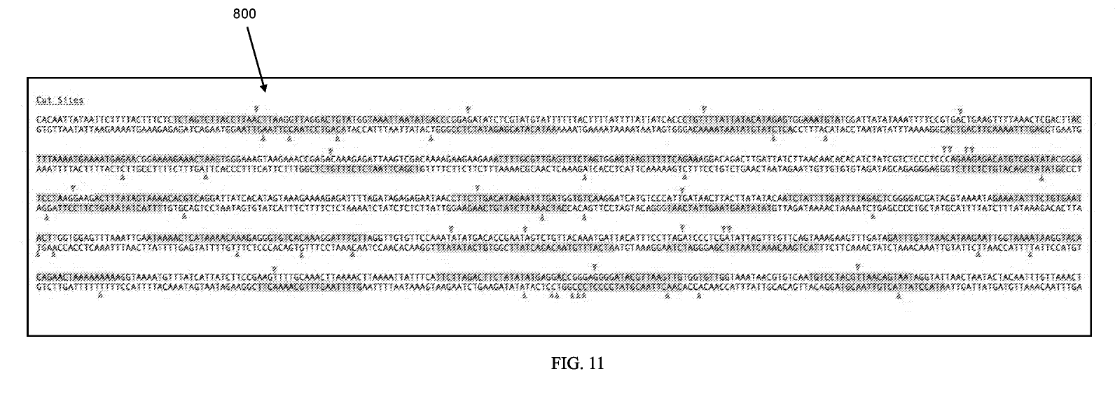

For the example, the candidate cut locations of the organism DNA sequence are illustrated in FIG. 11 and indicated by red arrows.

Example of Establishing Appropriate Cut Locations

Also as discussed above, a next step is to, for each candidate cut location, establish the candidate cut location as a respective appropriate cut location (see, e.g., reference 484) based on at least one cut location suitability factor, the at least one cut location suitability factor being one or more of a homology repair template maximum size, a homology repair template homology arm size, and a donor sequence maximum size.

Preferably, in this step, using the list of candidate cut locations, the action is to select the appropriate cut locations for cutting using some or all of the following criteria:

Maximum Size of the Entire Repair Template:

Factors to consider for this criterion preferably include, but are not limited to, the following:
Preferably, the maximum size of the repair template is derived by taking the delivery vehicle maximum cargo size, and subtracting the size of the targeted nuclease sequence (e.g., the nucleotide sequence that encodes the targeted nuclease), as well as the size of the targeting molecule (e.g., the molecule that is being used to find the target site) that contains the targeting sequences (e.g., guide sequences in this example); what remains is the available cargo space for the repair template.
The repair template is used after the targeted nuclease cuts the DNA; the cell repair machinery can attempt to repair the break through multiple repair pathways. In the presence of a repair template, one pathway used is homologous recombination, which is the pathway that is intended to be activated by having the repair template proximal to the targeted nuclease with the relevant targets when it cuts the DNA strand(s).
Preferably, a separate vehicle can be used to deliver the template if space considerations require it.
Preferably, other methods of ensuring proximity of the repair template with the correct targeted nuclease molecule (e.g., Cas9 and the like) and targeting molecule (e.g., the guide RNA or Zinc finger, and the like) can include covalently bonding the repair template to the targeted nuclease or to the targeting molecule.

Minimum Size of Homology Arms in Repair Template:

Factors to consider for this criterion preferably include, but are not limited to, the following:
Preferably, the minimum size is chosen based on the minimum desired level of homology between the repair template and the targeted DNA strand.
Preferably, the size and number of homology arms will depend on the selection of repair template type (single stranded, double stranded, plasmid and others) as well as the chosen cutting mechanism.

Maximum Size of Homology Arms in Repair Template:

Factors to consider for this criterion preferably include, but are not limited to, the following:
Preferably, the maximum size of the homology arms is chosen based on the maximum desired level of homology between the repair template and the target DNA strand.

Maximum Size of Repair Sequence:

Factors to consider for this criterion preferably include, but are not limited to, the following:
Preferably, the repair sequence maximum size is the amount of space left in the repair template after the minimum homology arm sequence length is subtracted.
Preferably, if there are two homology arms required in the repair template then both sequence lengths should be subtracted.

### Example

In the example, the desired criteria are as follows:
Maximum desired size of whole repair template: 500 bp
Minimum size of homology arms in repair template: 30 bp
Maximum size of homology arms in repair template: 120 bp
Maximum size of repair sequence: 440 bp
Overlap amount: 0%

### Example

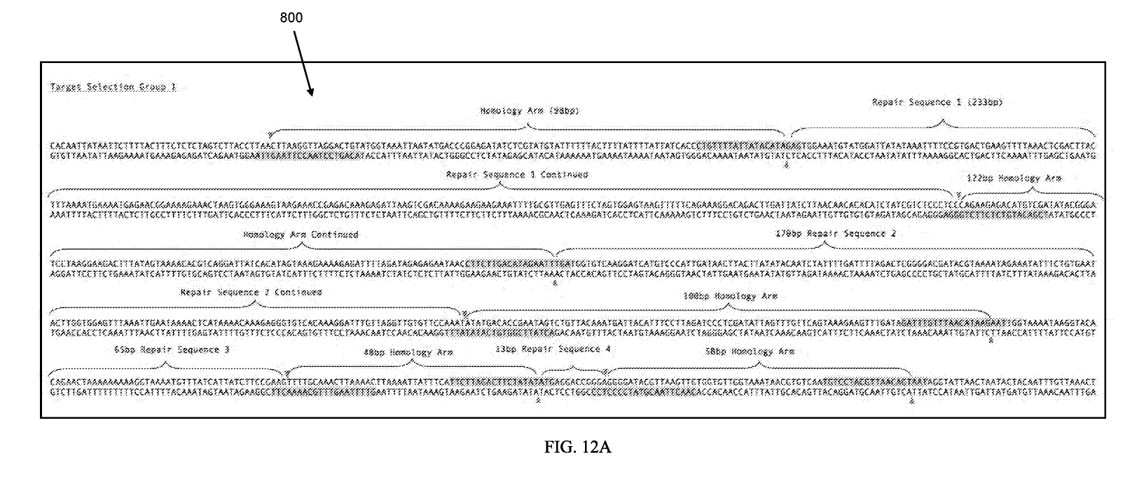

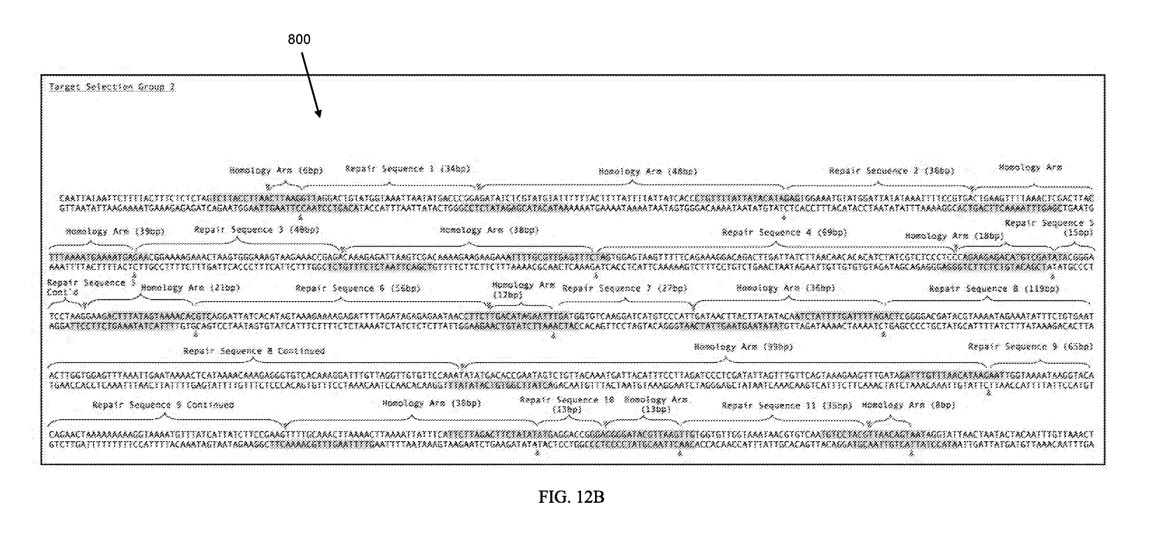

For the example, the appropriate cut locations of the organism DNA sequence are illustrated in FIG. 12A and indicated by red arrows.

Example of Establishing Homology Repair Templates

Also as discussed above, a next step is to establish a plurality of homology repair templates (see, e.g., reference 486) by, for each homology repair template, determining a homology repair template target group, the group including at least two cut locations of the appropriate cut locations, the group meeting at least one compatibility requirement with respect to at least one other homology repair template target group.

Preferably, in this step, the action is to select two (or more) cut locations that will serve as the first repair template target group. Preferably, the distance between the two or more cuts should not exceed the maximum desired size. Preferably, the maximum desired size can be the maximum repair sequence size calculated above, the cargo capacity of the delivery mechanism to be used, or another maximum size.

Preferably, to calculate if the cuts satisfy the maximum desired size requirement:
(a) group the cuts into the potential cutting group (the group of all cuts that will create the desired repair template);
(b) select the furthest upstream cut location of the group; depending on the targeted nuclease this will either be on 3′ or 5′ strand;
(c) select the furthest downstream cut location of the group; depending on the targeted nuclease this will be on the 3′ or 5′ strand;
(d) calculate the distance in base pairs (bp) between the two cut locations;
(e) if the distance is less than or equal to the maximum desired size, the template target group is valid;
(f) if the distance is greater than the maximum desired size, the template target group is not valid.

Further preferably, in this step, the next action is to select another two (or more) cut locations that will serve as the second repair template target group, preferably with the desired amount of overlap with the first template target group. Preferably, donor (e.g., repair) sequence overlap can be defined as when two or more repair sequences share base pair locations on the DNA strand. Preferably, repair template overlap can be defined as when two or more repair templates, including the homology arms, share the base pair locations on the DNA strand.

Preferably, to calculate repair sequence overlap:
(a) take the two outer-most cut locations in the first repair sequence; then take the two outer-most cut locations in the second repair sequence;
(b) the amount of shared base pairs is the amount of base pairs that are both contained between the outermost cut locations in the first repair sequence as well as the outer-most cut locations in the second repair sequence;
(c) the amount of overlap is the number of shared base pairs divided by the total number of non-shared base pairs multiplied by 100;
(d) an overlap of 0% means that there are no shared base pairs between both repair sequences;
(e) an overlap of 100% means that all base pairs are shared between both repair sequences;
(f) repeat this process for all repair sequences for which overlap is to be calculated.

Preferably, to calculate repair template overlap:
(a) take the two outer-most cut locations in the first repair template. Then take the two outer-most cut locations in the second repair template;
(b) the amount of shared base pairs is the amount of base pairs that are both contained between the outermost cut locations in the first repair template as well as the outer-most cut locations in the second repair template;
(c) the percentage of overlap is the number of shared base pairs divided by the total number of non-shared base pairs multiplied by 100;
(d) an overlap of 0% means that there are no shared base pairs between both repair templates;
(e) an overlap of 100% means that all base pairs are shared between both repair templates;
(f) repeat this process for all repair templates for which overlap is to be calculated.

Other factors to consider in selecting repair templates:
(a) uniqueness of homology arms to each other and to other sequences in the same or another repair template, target sequences and DNA strands;
(b) total number of cuts created in the organism DNA sequence;
(c) uniqueness of target sequences to each other and to other sequences in the same or another repair template, homology arms and DNA strands;
(d) presence of targeted nuclease binding site or any associated target sequences within repair template sequence.

Preferably, the above process is continued until the desired number of repair templates are established.

Further preferably, in this step, the next action is to store the location and sequence of the repair templates in a list of repair templates. Then, preferably, if desired or necessary, for each repair template, the next action is to mutate or remove any targeted nuclease binding site or target sequences present in the sequence that is the repair template to avoid unintended degradation by the targeted nuclease. Then, preferably, if desired or necessary, the next action is to, depending on the goal of the treatment, for each repair template, alter or replace the repair sequence to include the desired DNA sequence. Preferably, the homology arms can be modified, if required, to, for example, improve homologous recombination with the target DNA strand.

### Example

In the example, for the first repair template target group, it is preferred that the distance between the two cuts not exceed the maximum desired size of the whole repair template. Also in the example, for the second repair template target group, it is preferred that there be 0% overlap with the first repair template target group.

### Example

For the example, FIG. 12A shows four target selection groups that were chosen from the candidate sites in FIG. 11. Highlighted in brackets are the homology arm sequences and the repair sequences for each target selection group. Also for the example, FIG. 12B shows an alternative number of target selection groups created by changing the size/length requirements of the repair sequence and the homology arms during the process. However, the remainder of the discussions of the example will use the groups chosen as shown in FIG. 12A.

### Example

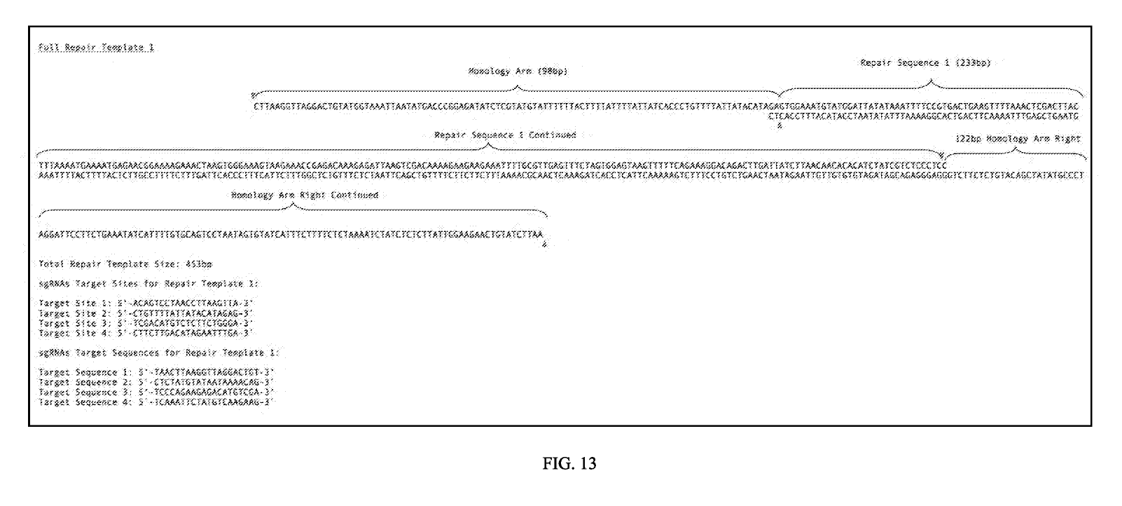

For the example, FIG. 13 shows the first homology repair template created by the first target selection group. Also shown is the total repair template size, as well as the target sites and the target sequences associated with this homology repair template.

### Example

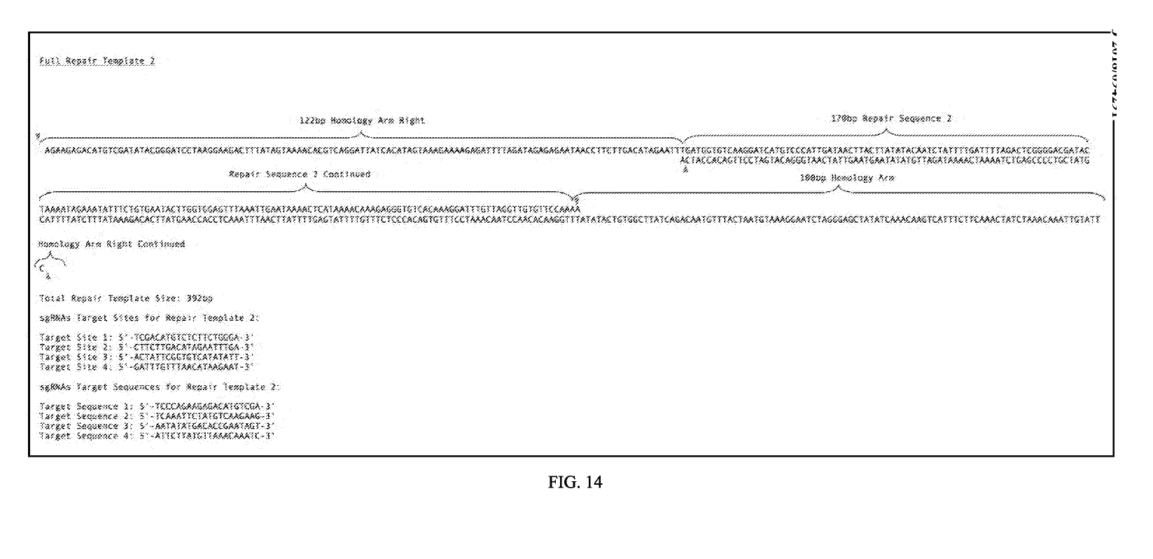

For the example, FIG. 14 shows the second homology repair template created by the second target selection group. Also shown is the total repair template size, as well as the target sites and the target sequences associated with this homology repair template.

### Example

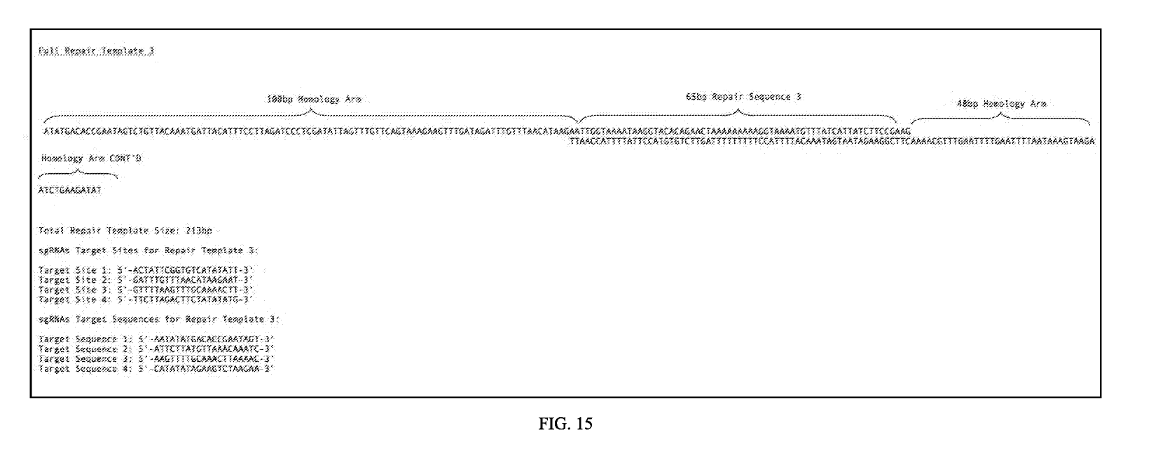

For the example, FIG. 15 shows the third homology repair template created by the third target selection group. Also shown is the total repair template size, as well as the target sites and the target sequences associated with this homology repair template.

### Example

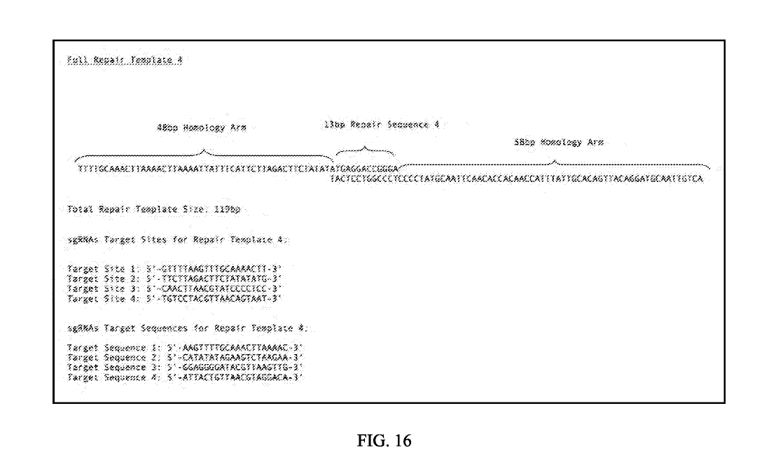

For the example, FIG. 16 shows the fourth homology repair template created by the fourth target selection group. Also shown is the total repair template size, as well as the target sites and the target sequences associated with this homology repair template.

Example of Establishing Dose Target Sites

Also as discussed above, a next step is to for each appropriate cut location, establish as a respective dose target site (see, e.g., reference 488) the candidate target site associated with the appropriate cut location.

Preferably, in this step, the action is to, for each cut location selected in the repair template target groups, look up the associated target site and store the associated target site in a list of dose target sites. (e.g., target sites that will be the sites, of the organism DNA sequence, targeted by the dose).

### Example

In the example, for each cut location selected in the four repair template target groups, the target site associated with the cut location is established as a dose target site.

Example of Translating Dose Target Sites into Targeting Sequences

Also as discussed above, a next step is to translate each dose target site into a respective targeted nuclease targeting sequence (see, e.g., reference 490).

Preferably, in this step, the action is to, for each target site in the list of dose target sites, translate the target sites into the targeted nuclease specific guide sequences, in any manner now known or hereafter developed or discovered.

### Example

In the example, the dose target sites are translated into the Cas9-variant specific guide sequences in a manner known in the art.

Once the dose target sites are translated into the guide sequences, other aspects of the invention are preferably undertaken, for example, as discussed herein with respect to FIG. 4C.

Candidate Targeted Nucleases

The following are targeted nucleases suitable for use with the present invention. It should be understood that other targeted nucleases, now known or hereafter developed and/or discovered, are also contemplated as suitable for use with the present invention.

SaCas9 Nickase

SpCas9: Streptococcus pyogenes

SpCas9n (D-10a)

SpCas9n (H840A)

dCas9

dCas9-Fokl

SaCas9

eSpCas9

SpCas9-HF1

HypaCas9

Cpf1

Zinc Finger Nuclease

Transcription activator-like effector Nuclease (TALEN)

### Acronyms and Terms

The following acronyms and terms as used herein are understood to have the following definitions, as complementary and/or supplementary to (and not as a replacement for) their definitions as known in the relevant art and/or to a skilled artisan in the relevant art:

CRISPR (Clustered Regular Interspaced Short Palindromic Repeats): a genetic engineering tool that uses a CRISPR sequence of DNA and its associated protein to edit the base pairs of a gene.

DNA (Deoxyribonucleic Acid): a self-replicating material present in nearly all living organisms as the main constituent of chromosomes. It is the carrier of genetic information.

RNA (Ribonucleic Acid): a nucleic acid present in all living cells. Its principal role is to act as a messenger carrying instructions from DNA for controlling the synthesis of proteins, although in some viruses RNA rather than DNA carries the genetic information.

ssODN (single stranded Oligodeoxynucleotide): a short sequence of nucleotides, whose nucleotides contain deoxyribose.

RNP (ribonucleoprotein): a nucleoprotein that contains RNA.

DSB (Double strand break): a break in the DNA in which both strands in the double helix are severed.

sgRNA (single guide RNA): a chimera of crRNA and tracrRNA that is typically 100 nucleotides in length and consists of three regions: (a) a user defined, 17-20nt base-pairing region for specific DNA binding; (b) a 40nt Cas9 handle hairpin for Cas9 protein binding; and (c) a 40nt long transcription terminator derived from S. pyogenes, that contains hairpin structures that provide stability to the RNA molecule.

Cas (CRISPR associated genes): RNA-guided DNA endonuclease enzyme associated with the CRISPR adaptive immunity system in Streptococcus pyogenes, among other bacteria.

NHEJ (Non-homologous end joining): a pathway that repairs double-strand breaks in DNA. NHEJ is referred to as “non-homologous” because the break ends are directly ligated without the need for a homologous template, in contrast to homology directed repair, which requires a homologous sequence to guide repair.

BER (Base Excision Repair): a cellular mechanism that repairs damaged DNA throughout the cell cycle. It is responsible primarily for removing small, non-helix-distorting base lesions from the genome.

DN (Double Nick): a discontinuity in a double stranded DNA molecule where there is no phosphodiester bond between adjacent nucleotides of both strands of the DNA molecule, typically through damage or enzyme action.

SN (Single Nick): A nick is a discontinuity in a double stranded DNA molecule where there is no phosphodiester bond between adjacent nucleotides of one strand typically through damage or enzyme action.

crRNA (CRISPR RNA): contains variable targeting sequence required for the Cas9 protein to target the DNA strand. crRNA forms a complex with tracrRNA to allow the Cas9 protein to bind to and cleave the DNA strand.

SaCas9 (Staphylococcus aures Cas9): Cas9 homologue found natively in Staphylococcus aureus bacteria.

SpCas9 (Staphylococcus pyogenes Cas9): Cas9 homologue found natively in Staphylococcus pyogenes bacteria.

tracrRNA (Trans-activating crRNA): a small trans-encoded RNA. It was first discovered in the human pathogen Streptococcus pyogenes. TracrRNA is complementary to and base pairs with a pre-crRNA forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

nDNA (Nuclear deoxyribonucleic acid): the DNA contained within the nucleus of a eukaryotic organism. Nuclear DNA encodes for the majority of the genome in eukaryotes, with mitochondrial DNA and plastid DNA coding for the rest.

AAV (Adeno-associated Virus): a small virus which infects humans and some other primate species. AAV is not currently known to cause disease. The virus causes a very mild immune response, lending further support to its apparent lack of pathogenicity. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise.

Donor sequence: Also referred to sometimes as a repair sequence, this is a sequence of DNA in, e.g., a homologous repair template, that is to replace a DNA sequence to be changed, e.g., as a result of a CRISPR event.

Additional Aspects of the Invention

It should be understood that the present invention encompasses the use of the described systems, devices and methods for partial or whole genome replacement in one or more cells of an organism, and not only in the medical fields, but also in other fields, and that the present invention is not limited to DNA sequence replacement, and that applications of the present invention to DNA sequence replacement are merely a subset of the possible embodiments of the present invention.

It should be understood that one or more systems and methods of the present invention preferably can be integrated with existing systems and methods and that any of such systems and methods of the present invention can be applied to effect partial or whole genome replacement in connection with one or more aspects of such systems and methods.

It should be understood that while certain methods are discussed herein as including preferred steps in a preferred order, it is contemplated that the present invention encompasses the steps being accomplished in other orders, not all of the steps being necessary, and/or one or more additional steps being taken, without departing from the scope of the present invention.

It should be understood that systems and methods described herein can be, but are not required to be, accomplished with or without the use of machines (including, but not limited to, with or without computers), and/or by one or more engines (such engines preferably including software running on at least one computer machine with a processor, memory, data storage capability, and networking capability, and preferably on two or more such computer machines communicating over a network such as, for example, the Internet), and, in certain embodiments, accomplished remotely, that is, over a network such as, for example, the Internet, an intranet, a wide area network, a local area network, and/or other network, through the operation of machines communicating with one another over the network, such as, for example, computers, tablets, smartphones, appliances, or any other network-enabled device. Further, calculations, transmissions and storage needed and/or employed by the present invention can be, but are not required to be, accomplished with secure calculation, transmission and storage protocols and using encryption and decryption protocols.

It should be understood that while arrows and lines on the drawings and illustrations have in certain instances been described herein as representing one direction of communication and/or activity, the present invention encompasses embodiments in which such lines of communication and/or activity are bi-directional or in the opposite direction than as indicated.

What is claimed is:

1. A method of substantially changing a DNA sequence of an organism, comprising the steps of:
determining a desired DNA sequence, the desired DNA sequence being a DNA sequence to which it is desired that the DNA sequence of the organism be substantially changed;
preparing a treatment configured to cause the organism DNA sequence to be substantially changed to the desired DNA sequence;
applying the treatment to the organism; wherein
the treatment is configured to cause the organism DNA sequence to be substantially changed to the desired DNA sequence by causing, at each of multiple sites in the organism DNA sequence, genetic code at the site to be substantially changed to genetic code at a corresponding site in the desired DNA sequence;
each of the multiple sites in the organism DNA sequence is a respective sub-sequence of the organism DNA sequence;
applying the treatment includes delivering to the organism at least one dose; and
each dose includes respective change agent material that causes the changing of the genetic code at a respective plurality of the multiple sites.

2. The method of claim 1, wherein the DNA sequence of the organism is a whole-genome DNA sequence of the organism.

3. The method of claim 1, wherein the desired DNA sequence is substantially at least one of a germline whole-genome DNA sequence of the organism, a pre-mutagenic whole-genome DNA sequence of the organism, a global average whole-genome DNA sequence of the organism, a whole-genome DNA sequence of another organism, and a whole-genome DNA sequence that is an intentionally modified version of a germline whole-genome DNA sequence of the organism.

4. The method of claim 1, wherein the desired DNA sequence is a desired whole-genome DNA sequence, the desired sequence having a plurality of nucleotides each at a respective nucleotide position in the desired sequence, the desired sequence being determined by a method comprising the steps of:
determining a necessary sample number, the necessary sample number being equal to a number of whole-genome DNA sequence samples, each having a plurality of nucleotides each at a respective nucleotide position in the sample, each sample being from a different cell of the organism, that must be sequenced to establish that, for each of the nucleotide positions in the desired sequence, the nucleotide at the corresponding nucleotide position in a majority of the samples is the nucleotide desireable for the nucleotide position in the desired sequence;
sequencing the necessary sample number of whole-genome DNA sequence samples to determine, for each of the samples, a respective sample sequence; and
establishing that, for each nucleotide position in the desired sequence, the nucleotide at the nucleotide position in the desired sequence is the nucleotide at the corresponding nucleotide position in a majority of the samples.

5. The method of claim 4, wherein a germline whole-genome DNA sequence of the organism has a plurality of nucleotides each at a respective nucleotide position in the germline sequence, and the nucleotide desireable for the nucleotide position in the desired sequence is the nucleotide in the corresponding nucleotide position in the germline sequence.

6. The method of claim 5, wherein the necessary sample number is equal to a number of the whole-genome DNA sequence samples that must be sequenced to establish that a probability, that for each sample, each nucleotide in its respective nucleotide position in the sample is not the nucleotide at the corresponding nucleotide position in the germline sequence, is less than a desired probability, the desired probability being no greater than one divided by the total number of nucleotides in the germline sequence.

7. The method of claim 6, wherein the desired probability is a product of a first factor and a second factor, the first factor being an error rate of a sequencing method used to sequence at least one of the samples, the second factor being a number no greater than one divided by the total number of nucleotides in the germline sequence.

8. The method of claim 4, wherein determining the necessary sample number includes accounting for an error rate of a sequencing method used to sequence at least one of the samples.

9. The method of claim 1, wherein preparing the treatment comprises one or more of the following steps:
determining, for each of the multiple sites, at least one targeting sequence;
determining, for each of the multiple sites, at least one homology repair template based on at least one characteristic of the at least one targeting sequence for the site;
synthesizing the targeting sequences and the homology repair templates;
amplifying the targeting sequences and the homology repair templates;
post-processing the homology repair templates;
packaging the targeting sequences and the homology repair templates into at least one delivery vehicle;
post-processing the delivery vehicle, wherein post-processing the delivery vehicle includes at least one of increasing delivery success and decreasing side effects;
preparing a solution that includes the change agent material, the change agent material including the post-processed delivery vehicle including the targeting sequences, the post-processed homology repair templates, and at least one of at least one targeted nuclease protein and at least one nucleic acid sequence that expresses at least one targeted nuclease protein; and
formulating the dose, the dose including the solution; wherein formulating the dose includes modifying, for treating the organism, the solution as to one or more of the following aspects: concentration, molarity, dosage and content.

10. The method of claim 9, wherein the targeting sequences and homology repair templates are determined by, the delivery vehicle is chosen by, and at least one of the at least one targeted nuclease protein and the at least one nucleic acid sequence are chosen by, repeating a process until all desired changes to the organism DNA sequence are expected to result from use of the change agent material in the dose, the process comprising one or more of the following steps:
for the dose, establish at least one dose requirement, the at least one dose requirement being based on at least one dose criterion, the at least one dose criterion being one or more of at least one delivery vehicle characteristic, at least one targeted nuclease characteristic, at least one off-target effect characteristic, at least one cutting mechanism characteristic, at least one organism characteristic, and at least one targeting sequence characteristic;
select a delivery vehicle and a targeted nuclease that meet the at least one dose requirement to a desired degree;
for each of a desired number of base pair sequences of the organism DNA sequence, when the targeted nuclease requires at least one binding site sequence specific to the targeted nuclease, compare the base pair sequence to the at least one binding site sequence;
for each of the compared base pair sequences, when the base pair sequence is compatible with the specific at least one binding site sequence, establish as a respective candidate binding site a binding site defined by binding site location information of the base pair sequence, the binding site location information including a start location, an end location, and a base pair letter sequence;
for each candidate binding site, using target site selection rules specific to the targeted nuclease, establish as a respective candidate target site a target site defined by target site location information associated with the candidate binding site, the target site location information including a start location and an end location, the rules including one or more of a distance from the candidate binding site and a base pair length;
for each candidate target site, using a cutting profile of the targeted nuclease, establish as a respective candidate cut location a location associated with the candidate target site and at which a cut is most likely to occur;
for each candidate cut location, establish the candidate cut location as a respective appropriate cut location based on at least one cut location suitability factor, the at least one cut location suitability factor being one or more of a homology repair template maximum size, a homology repair template homology arm size, and a donor sequence maximum size;
establish a plurality of homology repair templates by, for each homology repair template, determining a homology repair template target group, the group including at least two cut locations of the appropriate cut locations, the group meeting at least one compatibility requirement with respect to at least one other homology repair template target group;
for each appropriate cut location, establish as a respective dose target site the candidate target site associated with the appropriate cut location; and
translate each dose target site into a respective targeted nuclease targeting sequence.

11. The method of claim 10, wherein at least one of:
the at least one delivery vehicle characteristic is selected from the group consisting of a delivery vehicle size, a delivery vehicle cost, a delivery vehicle ease of ingestion, a delivery vehicle successful payload delivery likelihood, a delivery vehicle delivery route, and a delivery vehicle immunogenicity;
the at least one targeted nuclease characteristic is selected from the group consisting of a targeted nuclease targeting specificity, a targeted nuclease targeting consistency, a targeted nuclease binding specificity, a targeted nuclease binding consistency, a targeted nuclease cutting specificity, a targeted nuclease cutting consistency, targeted nuclease cost, a targeted nuclease required binding sequence distribution, a targeted nuclease required binding sequence probability, a targeted nuclease required binding sequence quantity;
the at least one off-target effect characteristic is selected from the group consisting of a likelihood of off-target effect occurrence, a likelihood of off-target effect amounts, and a likelihood of off-target cuts;
the at least one cutting mechanism characteristic is selected from the group consisting of a cutting mechanism type and a cutting mechanism action;
the at least one organism characteristic is selected from the group consisting of an organism nuclease activity amount and an organism nuclease degradation rate; and
the at least one targeting sequence characteristic is a targeting sequence length.

12. The method of claim 10, wherein the at least one compatibility requirement is selected from the group consisting of a homology repair template maximum size, a donor sequence overlap limit, a homology repair template overlap limit, a number of cuts to be made in the organism DNA sequence; a presence of a targeted nuclease binding site within a homology repair template sequence; and a presence of a donor sequence associated with a targeted nuclease binding site within a homology repair template sequence.

13. The method of claim 10, wherein the process further comprises, for at least one homology repair template, mutating or removing any targeted nuclease binding site sequences or donor sequences in the homology repair template.

14. The method of claim 10, wherein the process further comprises, for at least one homology repair template, modifying or replacing a donor sequence to include the desired DNA sequence.

15. The method of claim 10, wherein the targeted nuclease is CRISPR-Cas9.

16. The method of claim 1, wherein applying the treatment includes administering an aqueous solution to one or more cells of the organism.

17. The method of claim 16, wherein administering the solution includes at least one of injecting the solution into the organism adjacent to the one or more cells and intravenously introducing the solution into a bloodstream of the organism.

18. The method of claim 1, further comprising the steps of:
obtaining, from each of a plurality of cells of the organism, the cells being randomly chosen from among cells of interest, a DNA sequence from the cell;
comparing each of the obtained DNA sequences to the desired DNA sequence; and
based on results of the comparison, taking one or more of the following steps: applying the treatment to the organism again, applying to the organism a treatment modified based on results of the comparison, and determining that no additional treatment applications are necessary.

19. The method of claim 1, wherein the change agent material is useful to treat the organism but not useful to treat any other organism.

20. The method of claim 1, wherein the change agent material includes a plurality of targeting sequences, a plurality of homologous repair templates based on at least one of the plurality of targeting sequences, and at least one of at least one targeted nuclease protein and at least one nucleic acid sequence that expresses at least one targeted nuclease protein.


SEQUENCE LISTING (1 sequence)

SEQ ID NO: 1 - DNA, 995 bp, Homo sapiens
cacaattataattcttttactttctctctagtcttaccttaacttaaggttaggactgtatggtaaattaatatgacccggagatatctcgtatgtattttttacttttattttattatcaccctgttttattatacatagagtggaaatgtatggattatataaattttccgtgactgaagttttaaactcgacttactttaaaatgaaaatgagaacggaaaagaaactaagtgggaaagtaagaaaccgagacaaagagattaagtcgacaaaagaagaagaaattttgcgttgagtttctagtggagtaagtttttcagaaaggacagacttgattatcttaacaacacacatctatcgtctccctcccagaagagacatgtcgatatacgggatcctaaggaagactttatagtaaaacacgtcaggattatcacatagtaaagaaaagagattttagatagagagaataaccttcttgacatagaatttgatggtgtcaaggatcatgtcccattgataacttacttatatacaatctattttgattttagactcggggacgatacgtaaaatagaaatatttctgtgaatacttggtggagtttaaattgaataaaactcataaaacaaagagggtgtcacaaaggatttgttaggttgtgttccaaatatatgacaccgaatagtctgttacaaatgattacatttccttagatccctcgatattagtttgttcagtaaagaagtttgatagatttgtttaacataagaattggtaaaataaggtacacagaactaaaaaaaaaggtaaaatgtttatcattatcttccgaagttttgcaaacttaaaacttaaaattatttcattcttagacttctatatatgaggaccgggaggggatacgttaagttgtggtgttggtaaataacgtgtcaatgtcctacgttaacagtaataggtattaactaatactacaatttgttaaact